(19) United States
(12) Patent Application Publication
(10) Pub. No.: US 2016/0333397 A1
(43) Pub. Date: Nov. 17, 2016

(54) METHOD AND DEVICE FOR ANALYZING REACTION LIQUID AFTER NUCLEIC ACID AMPLIFICATION REACTION, AND DEVICE FOR PROCESSING REACTION LIQUID AFTER NUCLEIC ACID AMPLIFICATION REACTION

(71) Applicant: HITACHI, LTD., tokyo (JP)

(72) Inventors: Hiroko Matsunaga, Tokyo (JP); Tomoharu Kajiyama, Tokyo (JP); Mari Ohta, Tokyo (JP); Hideki Kambara, Tokyo (JP)

(73) Assignee: HITACHI, LTD., Tokyo (JP)

(21) Appl. No.: 15/112,493

(22) PCT Filed: Jan. 27, 2014
(86) PCT No.: PCT/JP2014/051633

§ 371 Date: Jul. 19, 2016

(51) Int. Cl. C12Q 1/68

(52) U.S. Cl. CPC C12Q 1/6816; C12Q 1/6874

(57) ABSTRACT

A reaction liquid after a nucleic acid amplification reaction is made suitable for various processes.

A step of measuring the amount of a target product and the amount of a byproduct after performing a nucleic acid amplification reaction, and a step of determining that a process for removing the byproduct is needed when the abundance ratio of the target product to the byproduct is lower than a prescribed value, and determining the dilution ratio of a reaction liquid after the nucleic acid amplification reaction when the abundance ratio is higher than the prescribed value are included.

### CROSS REFERENCE TO PRIOR APPLICATIONS

This application is a U.S. National Phase application under 35 U.S.C. §371 of International Application No. PCT/JP2014/051633, filed on Jan. 27, 2014. The International Application was published in Japanese on Jul. 30, 2015 as WO 2015/111209 A1 under PCT Article 21(2). The contents of the above applications are hereby incorporated by reference.

### SEQUENCE LISTING

The instant application contains a Sequence Listing which has been submitted electronically in ASCII format and is hereby incorporated by reference in its entirety. Pursuant to 37 C.F.R. §1.52 (e)(5), the Sequence Listing text file, identified as 072388_1268_Sequence_Listing.txt, is 1,245 bytes and was created on Jul. 12, 2016. The Sequence Listing, electronically filed herewith, does not extend beyond the scope of the specification and thus does not contain new matter.

### TECHNICAL FIELD

The present invention relates to an analysis method and an analysis device for analyzing a reaction liquid containing amplified fragments obtained by performing a nucleic acid amplification reaction using nucleic acids contained in a living body-derived sample as templates, and also relates to a reaction liquid processing device for processing a reaction liquid using the result of an analysis obtained by the analysis method and the analysis device.

### BACKGROUND ART

The monitoring of the expression level of a gene has been widely used for examining the function of the gene, examining the efficacy of a drug, making a diagnosis, or the like. In the monitoring, a technique for extracting mRNA from a cell, synthesizing cDNA which is a complementary strand to the mRNA, and measuring the cDNA is used. In order to perform a more detailed analysis, it is preferred to perform an analysis by subdividing the number of cells as much as possible, for example, to perform an analysis at a single cell level. However, when the number of cells to be used for an analysis is decreased, it is necessary to perform measurement after amplifying cDNA due to a problem of accuracy and detection sensitivity of a measurement device and the like. A general method for amplifying cDNA is a PCR method. Hereinafter, currently used techniques will be introduced.

First of all, a first method is a method in which mRNA is captured by a complementary binding reaction between a poly A sequence at the 3′ end of mRNA and an oligo(dT) DNA probe composed of a poly T sequence, and then, the oligo(dT) probe is elongated using the mRNA as a template, whereby a cDNA strand is obtained. In the case of this method, cDNA is synthesized from the 3′ end of mRNA, and therefore, a cDNA strand containing the 3′ end portion can be reliably obtained. Further, it is known that the poly A strand and the poly T strand of the capture probe slide and hybridize each other, and therefore, the capture efficiency is high.

A second method is a method in which a set of mixed primers composed of various sequences of about 6 to 9 bases called random primers is prepared, the random primers are complementarily bound to mRNA at a plurality of sites and elongated, whereby cDNA strands are obtained. In the case of this method, cDNA strands covering all the regions can be obtained regardless of the chain length of mRNA.

Further, preparation is sometimes performed by mixing an oligo(dT) probe and random primers using the first method and the second method in combination. In the case where a comprehensive analysis of genes expressed in cells or tissues is intended to be performed, a method capable of reliably obtaining the full length or a certain fixed portion, particularly a 3′ end portion which is considered to have a lot of gene-specific information (NPL 1: Cell (1985) Volume 41 349-359) is desirable, and a method using an oligo(dT) probe is used.

On the other hand, in a comprehensive gene analysis at a cell level or a tissue level, a technique by base sequence determination using a microarray method or a next generation sequencer is used. For such an analysis, a very large number of DNA samples are needed. Further, in the case where the amount of cDNA obtained from mRNA is extremely small, such as a case where mRNA extracted from a single cell is used, an analysis using a microarray method or a next generation sequencer is performed, and therefore, it is preferred to amplify cDNA in bulk. As the amplification method, PCR (Polymerase Chain Reaction) in which primers are complementarily bound to two sites sandwiching a region to be amplified, and the region to be amplified is repeatedly subjected to complementary strand synthesis so as to amplify the region is generally used. As a representative method for comprehensively amplifying all cDNA in a cDNA library at a single cell level, methods described in NPL 2: Nucleic Acids Research (2006) Volume 34, e42 and PTL 1: WO 06/085616 are known.

That is, the method is as follows. mRNA is captured by a probe (1) having a poly T sequence and an inherent sequence with abase length of about 20 bases at the 5′ end, and 1st strand cDNA is synthesized from the mRNA. Subsequently, a poly A sequence is introduced into the 3′ end of the synthesized 1st strand cDNA. A probe (2) having a poly T sequence complementary to this poly A sequence and an inherent sequence which is different from that of the probe (1) at the 5′ end is prepared, and by using the 1st strand cDNA as a template, cDNA is amplified in bulk by PCR using the probe (1) and the probe (2).

Such a method has a problem that also a byproduct is amplified. That is, the problem is such that when the poly A sequence is introduced into the 3′ end of the 1st strand cDNA, the poly A sequence is also introduced into the 3′ end of the remaining probe (1) in the same manner, and as a result, an amplification product (primer dimer) composed of only a primer sequence without including the cDNA portion is generated as a byproduct.

In general, in PCR, there is a tendency that a sequence having a shorter base length is more easily amplified, and therefore, the primer dimer is amplified in an overwhelmingly large excess amount as compared with cDNA. In order to prevent this, there is a method in which the generation of the byproduct is suppressed by degrading only a competitive primer in PCR before PCR using Exonuclease I which is a single strand specific DNase or by modifying the 5′ end of the competitive primer in the PCR with a phosphate group in advance and degrading only the primer using Lambda Exonuclease or the like which recognizes the phosphate group as an indicator and degrades the primer. Further, it is also possible to perform purification by fractionating a byproduct and a target product based on a difference in the molecular weight using a size fractionation column or the like.

### SUMMARY OF INVENTION

### Technical Problem

As described above, in the case where cDNA is amplified by utilizing a nucleic acid amplification reaction and an analysis is performed, or the like, a target amplified fragment and amplified fragments other than the target amplified fragment are sometimes intermingled with each other. Then, in the case where the amount of the amplified fragments other than the target amplified fragment is relatively large, there was a problem that a desired result cannot be obtained in, for example, an additional nucleic acid amplification reaction or an analysis such as base sequence determination, which is performed after the nucleic acid amplification reaction.

In view of such circumstances, an object of the present invention is to provide an analysis method and an analysis device, each of which makes a reaction liquid after a nucleic acid amplification reaction suitable for various processes by subjecting the obtained reaction liquid to an analysis after performing a nucleic acid amplification reaction using nucleic acids contained in a living body-derived sample as templates, and also another object thereof is to provide a reaction liquid processing device including the analysis device as well as the analysis method and the analysis device.

### Solution to Problem

The method for analyzing a reaction liquid after a nucleic acid amplification reaction according to the present invention which has achieved the above-mentioned objects includes a step of measuring the amount of a target amplified fragment and the amount of amplified fragments other than the target amplified fragment after performing a nucleic acid amplification reaction using nucleic acids contained in a living body-derived sample as templates, and a step of determining that a process for removing the amplified fragments other than the target amplified fragment is needed when the abundance ratio of the target amplified fragment to the amplified fragments other than the target amplified fragment is lower than a prescribed value, and determining the dilution ratio of a reaction liquid after the nucleic acid amplification reaction when the abundance ratio is higher than the prescribed value.

In the method for analyzing a reaction liquid after a nucleic acid amplification reaction according to the present invention, the method can be configured such that the nucleic acid amplification reaction is a nucleic acid amplification reaction using a carrier having an oligonucleotide composed of a poly T sequence corresponding to the poly A sequence of mRNA and a first inherent sequence immobilized thereon, and includes a step of capturing mRNA contained in the living body-derived sample to the carrier, a step of elongating a complementary strand to the mRNA from the poly T sequence, a step of adding a second inherent sequence to an end of the elongated strand, and a step of performing amplification using a first primer having a complementary sequence to the first inherent sequence and a second primer having a complementary sequence to the second inherent sequence.

Further, in the method for analyzing a reaction liquid after a nucleic acid amplification reaction according to the present invention, in the determination step, it can be determined that the dilution ratio is 1 when the amount of the target amplified fragment is higher than a first prescribed value. That is, it can be determined that when the amount of the target amplified fragment is higher than the first prescribed value, for example, the reaction liquid can be used for a process for analyzing the target amplified fragment without performing a process for removing the amplified fragments other than the target amplified fragment from the reaction liquid or a process for diluting the reaction liquid. In this case, the first prescribed value can be defined as the lower limit value of the amount of the amplified fragment to be used in the analysis process.

In addition, in the analysis method according to the present invention, in the determination step, the ratio (A/B) calculated from the amount (A) of the target amplified fragment and the amount (B) of the amplified fragments other than the target amplified fragment and the ratio (X/Y) of a second prescribed value (X) determined for the amount (A) of the target amplified fragment to a third prescribed value (Y) determined for the amount (B) of the amplified fragments other than the target amplified fragment are compared, and it can be determined that the reaction liquid after the nucleic acid amplification reaction is diluted to (A/X) times when the ratio (A/B) is larger than the ratio (X/Y) and also the amount (A) of the target amplified fragment is larger than the second prescribed value (X); it can be determined that the dilution ratio of the reaction liquid after the nucleic acid amplification reaction is 1 when the ratio (A/B) is larger than the ratio (X/Y) and also the amount (A) of the target amplified fragment is smaller than the second prescribed value (X); it can be determined that the reaction liquid after the nucleic acid amplification reaction is diluted to (B/Y) times when the ratio (A/B) is smaller than the ratio (X/Y) and also the amount (B) of the amplified fragments other than the target amplified fragment is larger than the third prescribed value (Y); and it can be determined that the dilution ratio of the reaction liquid after the nucleic acid amplification reaction is 1 when the ratio (A/B) is smaller than the ratio (X/Y) and also the amount (B) of the amplified fragments other than the target amplified fragment is smaller than the third prescribed value (Y). That is, in the determination step, by setting the second prescribed value (X) and the third prescribed value (Y), the amount of the target amplified fragment and/or the amount of the amplified fragments other than the target amplified fragment in the reaction liquid can be adjusted to, for example, an optimal amount for an additional amplification reaction for the target amplified fragment. In this case, the second prescribed value can be defined as the upper limit value of the amount of the target amplified fragment contained in the reaction liquid to be used for the additional amplification reaction. Further, the third prescribed value can be defined as the upper limit value of the amplified fragments other than the target amplified fragment contained in the reaction liquid to be used for the additional amplification reaction.

On the other hand, an analysis device according to the present invention includes a measurement section which measures the amount of a target amplified fragment and the amount of amplified fragments other than the target amplified fragment contained in a reaction liquid after performing a nucleic acid amplification reaction using nucleic acids contained in a living body-derived sample as templates, and a determination section which determines that a process for removing the amplified fragments other than the target amplified fragment is needed when the abundance ratio of the target amplified fragment to the amplified fragments other than the target amplified fragment is lower than a prescribed value, and determines the dilution ratio of the reaction liquid after the nucleic acid amplification reaction when the abundance ratio is higher than the prescribed value based on the value measured by the measurement section.

Further, in the analysis device according to the present invention, the measurement section can measure the amount of the target amplified fragment and the amount of the amplified fragments other than the target amplified fragment in the reaction liquid after the nucleic acid amplification reaction which is a nucleic acid amplification reaction using a carrier having a poly T sequence corresponding to the poly A sequence of mRNA and a first prescribed sequence immobilized thereon, and includes a step of capturing mRNA contained in the living body-derived sample to the carrier, a step of elongating a complementary strand to the mRNA from the poly T sequence, a step of adding a second prescribed sequence to an end of the elongated strand, and a step of performing amplification using a first primer having a complementary sequence to the first prescribed sequence and a second primer having a complementary sequence to the second prescribed sequence.

In addition, in the analysis device according to the present invention, in the determination section, it can be determined that the dilution ratio is 1 when the amount of the target amplified fragment is higher than a first prescribed value. That is, in the analysis device according to the present invention, it can be determined that when the amount of the target amplified fragment is higher than the first prescribed value, for example, the reaction liquid can be used for a process for analyzing the target amplified fragment without performing a process for removing the amplified fragments other than the target amplified fragment from the reaction liquid or a process for diluting the reaction liquid. In this case, the first prescribed value can be defined as the lower limit value of the amount of the amplified fragment to be used in the analysis process.

Still further, in the analysis device according to the present invention, in the determination section, the ratio (A/B) calculated from the amount (A) of the target amplified fragment and the amount (B) of the amplified fragments other than the target amplified fragment and the ratio (X/Y) of a second prescribed value (X) determined for the amount (A) of the target amplified fragment to a third prescribed value (Y) determined for the amount (B) of the amplified fragments other than the target amplified fragment are compared, and it can be determined that the reaction liquid after the nucleic acid amplification reaction is diluted to (A/X) times when the ratio (A/B) is larger than the ratio (X/Y) and also the amount (A) of the target amplified fragment is larger than the second prescribed value (X); it can be determined that the dilution ratio of the reaction liquid after the nucleic acid amplification reaction is 1 when the ratio (A/B) is larger than the ratio (X/Y) and also the amount (A) of the target amplified fragment is smaller than the second prescribed value (X); it can be determined that the reaction liquid after the nucleic acid amplification reaction is diluted to (B/Y) times when the ratio (A/B) is smaller than the ratio (X/Y) and also the amount (B) of the amplified fragments other than the target amplified fragment is larger than the third prescribed value (Y); and it can be determined that the dilution ratio of the reaction liquid after the nucleic acid amplification reaction is 1 when the ratio (A/B) is smaller than the ratio (X/Y) and also the amount (B) of the amplified fragments other than the target amplified fragment is smaller than the third prescribed value (Y). That is, in the determination section of the analysis device according to the present invention, by setting the second prescribed value (X) and the third prescribed value (Y), the amount of the target amplified fragment and/or the amount of the amplified fragments other than the target amplified fragment in the reaction liquid can be adjusted to, for example, an optimal amount for an additional amplification reaction for the target amplified fragment. In this case, the second prescribed value can be defined as the upper limit value of the amount of the target amplified fragment contained in the reaction liquid to be used for the additional amplification reaction. Further, the third prescribed value can be defined as the upper limit value of the amplified fragments other than the target amplified fragment contained in the reaction liquid to be used for the additional amplification reaction.

On the other hand, the analysis device according to the present invention described above can constitute as a part of a device for processing a reaction liquid after a nucleic acid amplification reaction including a dilution processing section which performs a dilution process for a reaction liquid after performing a nucleic acid amplification reaction according to the determination made by the determination section. That is, the reaction liquid processing device includes the analysis device according to the present invention described above and a dilution processing section which performs a dilution process for a reaction liquid after performing a nucleic acid amplification reaction according to the determination made by the determination section.

Further, the reaction liquid processing device according to the present invention may further include a nucleic acid amplification reaction processing section which performs an additional nucleic acid amplification reaction using the reaction liquid having been subjected to the dilution process in the dilution processing section or the reaction liquid having not been subjected to the dilution process.

In addition, the reaction liquid processing device according to the present invention may further include a sequence determination processing section which determines the base sequence of a target amplified fragment in a reaction liquid for which it has been determined that the dilution ratio is 1 based on the fact that the amount of the target amplified fragment is higher than a first prescribed value in the determination section in the analysis device or a reaction liquid after performing a nucleic acid amplification reaction in the nucleic acid amplification reaction processing section.

### Advantageous Effects of Invention

According to the present invention, a reaction liquid after performing a nucleic acid amplification reaction using nucleic acids contained in a living body-derived sample as templates can be made suitable for various processes. That is, by using the analysis method and the analysis device according to the present invention, a reaction liquid after performing a nucleic acid amplification reaction can be made suitable for various processes by determining the necessity of a process for removing the amplified fragments other than the target amplified fragment with respect to the reaction liquid and the dilution ratio of the reaction liquid after the nucleic acid amplification reaction.

Further, the reaction liquid processing device according to the present invention can perform a process for diluting a reaction liquid after performing a nucleic acid amplification reaction to a suitable concentration for various processes. Therefore, the reaction liquid processing device according to the present invention can accurately perform various processes.

### BRIEF DESCRIPTION OF DRAWINGS

FIG. 1 is a flowchart schematically showing a step of producing a cDNA library using a magnetic bead.

FIG. 2 is a view schematically showing the generation of a byproduct in cDNA produced using a magnetic bead.

FIG. 3 is a flowchart showing one embodiment of an analysis method according to the present invention.

FIG. 4 is a view showing prescribed values to be used in one embodiment of the analysis method according to the present invention.

FIG. 5 is a structural view schematically showing one embodiment of an analysis device according to the present invention.

FIG. 6 is a structural view schematically showing another embodiment of the analysis device according to the present invention.

FIG. 7 is a structural view schematically showing one embodiment of a reaction liquid processing device according to the present invention.

FIG. 8 is a structural view schematically showing another embodiment of the reaction liquid processing device according to the present invention.

FIG. 9A is a structural view schematically showing another embodiment of the reaction liquid processing device according to the present invention.

FIG. 9B is a structural view schematically showing another embodiment of the reaction liquid processing device according to the present invention.

FIG. 10 is a characteristic view showing a relationship between a nucleic acid sample with a known concentration and a peak area obtained from an electropherogram.

FIG. 11 is a characteristic view showing the result of electrophoresis performed for a reaction liquid after a nucleic acid amplification reaction performed in Example.

FIG. 12 is a characteristic view showing the result of electrophoresis performed after a purification process for a reaction liquid after a nucleic acid amplification reaction performed in Example.

FIG. 13 is a characteristic view showing the result of electrophoresis of an amplification product having been subjected to second purification performed in Example.

FIG. 14 is a characteristic view showing the result of electrophoresis performed after a first amplification product was purified twice in Example.

FIG. 15 is a characteristic view showing the result of electrophoresis performed after the first amplification product was purified twice and thereafter further purified in Example.

FIG. 16 is a characteristic view showing a relationship between the amounts of a target product and a byproduct calculated from the result of electrophoresis and whether or not sequence data derived from cDNA are obtained.

FIG. 17 is a characteristic view showing the result of electrophoresis performed after the first amplification product was purified twice in Example.

FIG. 18 is a characteristic view showing the result of electrophoresis performed after a product of a first amplification reaction performed using a template diluted to 1/10 was purified twice.

FIG. 19 is a characteristic view showing the result of a comprehensive analysis performed by a next generation sequencer using two samples of an amplification product which was not diluted.

FIG. 20 is a characteristic view showing the result of a comprehensive analysis performed by a next generation sequencer using two samples of an amplification product which was diluted to 1/10.

FIG. 21 is a characteristic view showing R value, X value, Y value, and F value calculated from the result of Example.

### DESCRIPTION OF EMBODIMENTS

Hereinafter, the present invention will be described in detail with reference to the drawings.

A method for analyzing a reaction liquid after a nucleic acid amplification reaction (hereinafter simply referred to as “analysis method”), a device for analyzing a reaction liquid after a nucleic acid amplification reaction (hereinafter simply referred to as “analysis device”), and a reaction liquid processing device to which the present invention is applied perform an analysis process for a reaction liquid containing a “target amplified fragment” and “amplified fragments other than the target amplified fragment” obtained by a nucleic acid amplification reaction. Here, the term “target amplified fragment” refers to a nucleic acid fragment obtained by correct annealing of primers to a template so as to amplify a desired region of the template among the nucleic acid fragments amplified by a nucleic acid amplification reaction performed in a reaction system containing the template, the primers, bases serving as substrates, and a nucleic acid synthase. The term “amplified fragments other than the target amplified fragment” refers to nucleic acid fragments other than the “target amplified fragment” defined as described above among the nucleic acid fragments amplified by a nucleic acid amplification reaction. Examples of the “amplified fragments other than the target amplified fragment” can include nucleic acid fragments resulted from annealing of the primers to nucleic acids present as contaminants different from the template so as to amplify a region other than the desired region.

Further, in the analysis method and the analysis device, the nucleic acid amplification reaction is not particularly limited, and may be a nucleic acid amplification reaction with any principle or mechanism. In particular, the nucleic acid amplification reaction is preferably a nucleic acid amplification reaction which amplifies a plurality of different regions in bulk. In this case, nucleic acid fragments amplified from the plurality of regions are “target amplified fragments”, and nucleic acid fragments other than these fragments are “amplified fragments other than the target amplified fragments”.

Examples of the nucleic acid amplification reaction which amplifies a plurality of different regions in bulk can include a nucleic acid amplification reaction which performs bulk amplification using a lot of cDNA synthesized by reverse transcription from a lot of mRNA extracted from cells or tissues as templates. However, the nucleic acid amplification reaction which amplifies a plurality of different regions in bulk is not limited to this example, and for example, a nucleic acid amplification reaction in which an adaptor is ligated to a lot of nucleic acid fragments obtained by treating a genome extracted from cells or tissues with a restriction enzyme, and bulk amplification is performed by using the lot of nucleic acid fragments as templates using primers which specifically anneal to the adaptor can be exemplified. In all the nucleic acid amplification reactions described above, amplification is performed by PCR, however, additional examples of the nucleic acid amplification reaction which amplifies a plurality of different regions in bulk include a method in which random primers are bound to DNA to be used as a template, and complementary strands are synthesized by a DNA polymerase having a strand displacement activity, and also complementary strands are further synthesized while being replaced by a newly synthesized strand. However, in general, the efficiency of amplification by strand displacement is very low as compared with that of PCR.

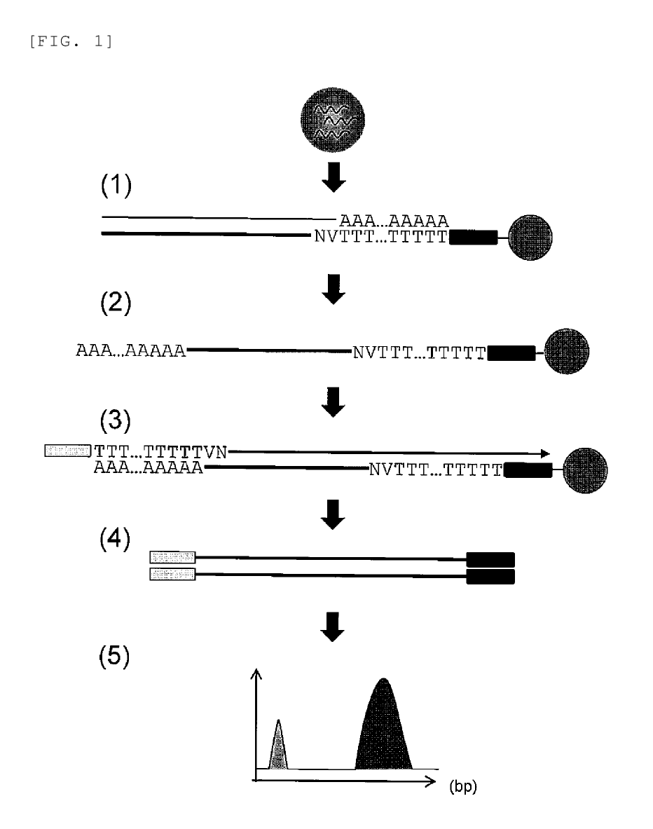

More specific examples of the nucleic acid amplification reaction which amplifies a plurality of different regions in bulk can include a nucleic acid amplification reaction as shown in FIG. 1. Specifically, first, as shown in FIG. 1(1), mRNA extracted from a biological sample such as cells is captured using a probe for capturing mRNA immobilized on a solid phase carrier (here, a magnetic bead is used) beforehand, and cDNA is synthesized by a reverse transcription reaction on the solid phase. Subsequently, as shown in FIG. 1(2), a poly A sequence is introduced into the 3′ end of the synthesized cDNA. Subsequently, as shown in FIG. 1(3), a double-stranding reaction of cDNA is performed using a primer for synthesizing double-stranded cDNA having a poly T sequence complementary to the poly A sequence introduced into the 3′ end of the cDNA. Then, as shown in FIG. 1(4), bulk amplification of nucleic acids is performed by using the synthesized double-stranded cDNA as a template. In this manner, according to the method shown in FIGS. 1(1) to 1(4), a lot of cDNA corresponding to a lot of mRNA extracted from a biological sample is amplified in bulk.

Therefore, in the method shown in FIGS. 1(1) to 1(4), the term “target amplified fragment” refers to a lot of cDNA amplified in bulk corresponding to a lot of mRNA. Then, in the method shown in FIGS. 1(1) to 1(4), nucleic acid fragments are amplified other than the lot of cDNA amplified in bulk corresponding to the lot of mRNA. The amplified fragments contained in the reaction liquid after the nucleic acid amplification reaction shown in FIG. 1(4) are separated according to the base length, and the amount thereof is measured. As a result, for example, as shown in FIG. 1(5), a peak corresponding to the lot of cDNA and a peak corresponding to the amplified fragments other than the cDNA, that is, the “amplified fragments other than the target amplified fragments” are detected.

The method shown in FIGS. 1(1) to 1(4) will be described in more detail. On the magnetic bead to be used in this method, a probe for capturing mRNA is immobilized beforehand. Examples of the probe for capturing mRNA include SEQ ID NO: 1: 5′-ATATGGATCCGGCGCGCCGTCGACTTTTTTTTTTTTTTTTTTTTTTTTVN-3′ (here, V=a mixed base of A, C, or G, N=a mixed base of A, C, G, or T). On the magnetic bead, the probe for capturing mRNA is immobilized at the 5′ end. A sequence on the 5′ end side of the probe for capturing mRNA shown in this example is a first inherent sequence (24 bases on the 5′ end side in SEQ ID NO: 1) corresponding to a primer sequence 1 for PCR to be used in a subsequent nucleic acid amplification step. Further, the probe for capturing mRNA shown in this example on the 3′ end side has a poly T sequence (from the 25th-base to the 48th-base counted from the 5′ end side in SEQ ID NO: 1) corresponding to the poly A sequence of mRNA and a sequence (2 bases on the 3′ end side in SEQ ID NO: 1) corresponding to the sequence upstream from the poly A sequence of mRNA. The probe for capturing mRNA can capture mRNA by complementarily binding the poly T sequence on the 3′ end side and the sequence corresponding to the sequence upstream from the poly A sequence of the mRNA of the probe to mRNA. Incidentally, since the probe has a sequence (VN in SEQ ID NO: 1) corresponding to the sequence upstream from the poly A sequence of mRNA, a base immediately upstream of the poly A sequence of mRNA can be used as the start site of the reverse transcription reaction.

Incidentally, the sequence of the probe for capturing mRNA is not limited to SEQ ID NO: 1. The first inherent sequence on the 5′ end side can be appropriately designed according to the PCR primer sequence 1 to be used in a subsequent nucleic acid amplification reaction. Incidentally, the base length of the first inherent sequence is also arbitrary and can be set to, for example, a base length of about 15 to 30 bases according to the PCR primer sequence 1 to be used in a subsequent nucleic acid amplification reaction. Further, the number of T bases of the poly T sequence on the 3′ end side is also not particularly limited, and the base length may be set to 24 bases as the example of the sequence represented by SEQ ID NO: 1. However, the base length is not limited thereto and can be set to, for example, 12 to 40 bases. When the number of T bases in the poly T sequence is within this range, mRNA can be reliably captured.

Further, in the above-mentioned example, as the solid phase having the probe for capturing mRNA immobilized thereon, a magnetic bead is shown, however, the material of the solid phase carrier is not particularly limited thereto. The solid phase carrier is not particularly limited as long as it is not soluble in water, and examples thereof include metals such as gold, silver, copper, aluminum, platinum, titanium, and nickel, alloys such as stainless steel and duralumin, glass materials such as silicon, glass, quartz glass, and ceramics, plastics such as polyester resins, polystyrene, polypropylene resins, nylon, epoxy resins, and vinyl chloride resins, agarose, dextran, cellulose, polyvinyl alcohol, and chitosan. Further, the shape of the carrier is not limited to a spherical shape, and may be any shape such as a plane, a titer plate, or a porous membrane.

Further, the amount of the probe for capturing mRNA immobilized on the surface of the magnetic bead is not particularly limited, but it is preferred that the probe is immobilized in a large excess amount (about 10³ to 10⁸ times) as compared with the number of molecules of mRNA to be captured actually. By setting the amount of the probe for capturing mRNA immobilized on the surface of the magnetic bead within the above range with respect to the number of molecules of mRNA to be captured, the efficiency of capture of mRNA is improved.

First, the step of FIG. 1(1) will be described. mRNA is captured on the magnetic bead by complementary binding between the poly A sequence at the 3′ end of mRNA eluted from a cell and the poly T sequence of the capture probe immobilized on the magnetic bead. Although the contents of mRNA vary depending on the biological species, tissue, or organ, in general, in the case of a human being, about 10,000 to 20,000 types of mRNA per cell are expressed in various copy numbers, respectively, and about 10⁵ copies in total are considered to be expressed. The base length and base sequence of the mRNA also vary. Subsequently, by using the captured mRNA as a template, cDNA is synthesized by a reverse transcription reaction from the 3′ end side of the probe for capturing mRNA. After the reverse transcription reaction, the magnetic bead is captured with a magnet and separated from the reaction solution, and the components of the reaction solution are removed by washing the magnetic bead. At this time, by performing the washing operation a plurality of times, it is possible to prevent the carry-over of the reaction solution of the previous step which becomes a factor of inhibiting the reaction in a subsequent step.

Subsequently, as shown in FIG. 1(2), a sequence composed of a plurality of A bases is inserted by a poly A tailing reaction into the end of the synthesized cDNA. At this time, the sequence composed of a plurality of A bases (poly A sequence) is inserted not only into the synthesized cDNA, but also into the 3′ end of the excess probe for capturing mRNA which were immobilized on the magnetic bead and did not contribute to the synthesis of cDNA. After the poly A tailing reaction, the magnetic bead is captured again with a magnet and separated from the reaction solution, and the components of the reaction solution of the poly A tailing reaction are removed by washing the magnetic bead. In the same manner as described above, by performing the washing operation a plurality of times, it is possible to prevent the carry-over of the solution of the previous step which becomes a cause of inhibiting the reaction in a subsequent step.

Subsequently, as shown in FIG. 1(3), a complementary strand is synthesized by an elongation reaction using a primer for synthesizing double-stranded cDNA having a poly T sequence complementary to the poly A sequence introduced into the 3′ end of the cDNA and also using the single-stranded cDNA as a template (a 2nd strand synthesis reaction). In this example, as the primer for synthesizing double-stranded cDNA, for example, SEQ ID NO: 2: 5′-ATATCTCGAGGGCGCGCCGGATCCTTTTTTTTTTTTTTTTTTTTTTTTVN-3′ (here, V=a mixed base of A, C, or G, N=a mixed base of A, C, G, or T) can be used. A sequence on the 5′ end side of the primer for synthesizing double-stranded cDNA shown in this example is a second inherent sequence (24 bases on the 5′ end side in SEQ ID NO: 2) corresponding to a primer sequence 2 for PCR to be used in a subsequent nucleic acid amplification step. Further, the primer for synthesizing double-stranded cDNA shown in this example on the 3′ end side has a poly T sequence (from the 25th-base to the 48th-base counted from the 5′ end side in SEQ ID NO: 2) corresponding to the poly A sequence of mRNA and a sequence (2 bases on the 3′ end side in SEQ ID NO: 2) corresponding to the sequence immediately upstream of the poly A sequence introduced into the cDNA, that is, the sequence at the end of the synthesized cDNA. The primer for synthesizing double-stranded cDNA can synthesize a complementary strand using the single-stranded cDNA as a template by complementarily binding the poly T sequence on the 3′ end side of the primer and the sequence corresponding to the sequence at the end of the cDNA to the introduced poly A sequence and the sequence at the end of the single-stranded cDNA, respectively.

Incidentally, the primer for synthesizing double-stranded cDNA is not limited to the base sequence represented by SEQ ID NO: 2. The second inherent sequence on the 5′ end side can be appropriately designed according to the PCR primer sequence 2 to be used in a subsequent nucleic acid amplification reaction. However, the second inherent sequence may be a sequence which hardly forms a secondary structure with the first inherent sequence located on the 5′ end side of the probe for capturing mRNA, and also is desirably a sequence which has a Tm value (melting temperature) close to that of the first inherent sequence. The phrase “Tm values close to each other” refers to that the difference in Tm is desirably within about ±10° C., more desirably within about ±5° C. Incidentally, it is also possible that the first inherent sequence and the second inherent sequence are composed of the same base sequence. In addition, the number of T bases of the poly T sequence on the 3′ end side of the primer for synthesizing double-stranded cDNA is also not particularly limited, and the base length may be set to 24 bases as the example of the sequence represented by SEQ ID NO: 2. However, the base length is not limited thereto, and can be set to, for example, 12 to 40 bases. Further, since the primer for synthesizing double-stranded cDNA has a sequence (VN in SEQ ID NO: 2) corresponding to the sequence at the end of the synthesized cDNA, only the primer bound to the end of the single-stranded cDNA (a site adjacent to the poly A sequence in the cDNA sequence on the most upstream side of the introduced poly A portion) can undergo an elongation reaction. It is difficult to control the number of A bases to be inserted by the above-mentioned poly A tailing reaction, and the base length is considered to be from several tens to several hundreds of bases. If the primer for synthesizing double-stranded cDNA does not have the sequence corresponding to the sequence at the end of the cDNA at the 3′ end thereof, the poly T portion of the primer for synthesizing double-stranded cDNA can complementarily bind to any site of this poly A sequence in a wide range, and also an elongation reaction may start. Further, a plurality of primers may complementarily bind to one cDNA and an elongation reaction may start from each primer, and therefore, unnecessary byproducts may be generated. Since the primer for synthesizing double-stranded cDNA has the sequence corresponding to the sequence at the end of the synthesized cDNA, these factors can be eliminated.

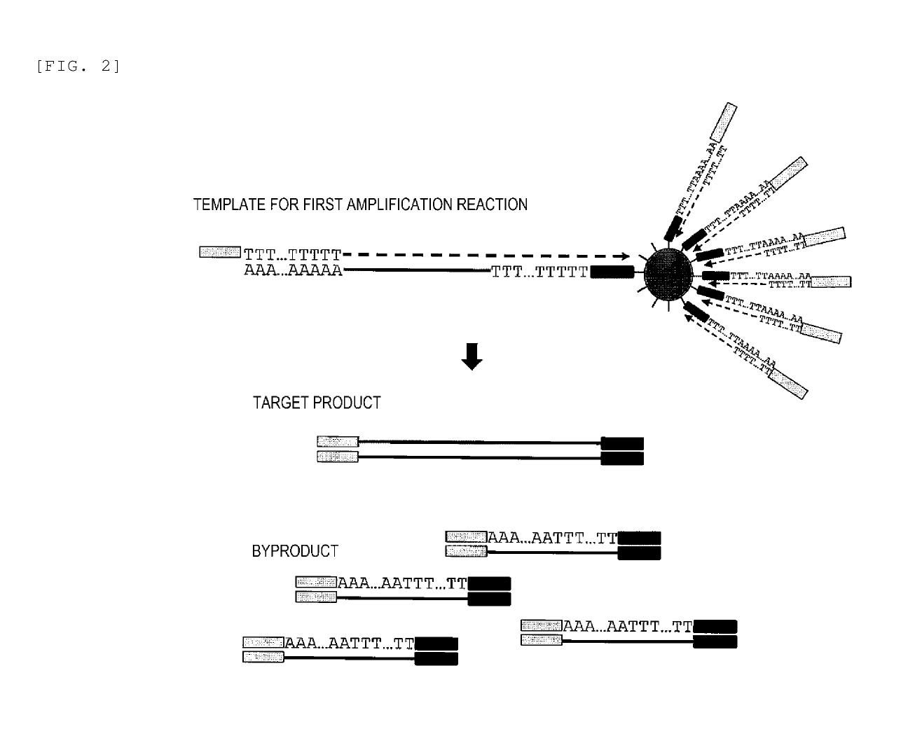

In the step shown in FIG. 1(3), a cDNA library which is immobilized on the magnetic bead at one end thereof and has the first inherent sequence and the second inherent sequence (corresponding to the primer sequences 1 and 2 for PCR) on both ends can be constructed. Subsequently, as shown in FIG. 1(4), by a nucleic acid amplification reaction utilizing the first inherent sequence and the second inherent sequence, the cDNA library is amplified in bulk. At this time, as shown in FIG. 2, a nucleic acid amplification reaction proceeds also between the first inherent sequence in the probe for capturing mRNA which did not contribute to the synthesis of cDNA and the second inherent sequence of the primer for synthesizing double-stranded cDNA annealed to the poly A sequence introduced into the probe for capturing mRNA. As a result, a so-called primer dimer is amplified other than the target amplified fragments.

As described in detail above, after a nucleic acid amplification reaction is performed using nucleic acids contained in a living body-derived sample as templates, a reaction liquid containing target amplified fragments (hereinafter, sometimes also referred to as “target products”) and amplified fragments other than the target amplified fragments (hereinafter, sometimes also referred to as “byproducts”) is obtained. The target products contained in this reaction liquid are not particularly limited, and are subjected to various analysis processes. As one example of the analysis process for the target products, an analysis process in which the target products contained in the reaction liquid are sequenced using a so-called next generation sequencer can be exemplified. Here, the “next generation sequencer” is also called “second generation sequencer” and is a base sequence determination device for determining the base sequences of several tens of millions of DNA fragments simultaneously and in parallel. The next generation sequencer is not particularly limited, however, for example, a device (Illumina, Inc.) which performs amplification of a DNA fragment on a slide glass called “flow cell”, and adopts a principle that the sequence is determined while synthesizing a complementary strand to the formed fragment can be exemplified.

For example, for the preparation of a library for an analysis using the next generation sequencer, cDNA (target product) in an amount of several tens of ng to several μg is needed. For example, the amount of mRNA derived from a single cell is from 0.1 pg to several pg, and an amplification step becomes essential for the analysis using the next generation sequencer. In general, as the PCR cycle number is increased, a bias due to a competitive reaction in the mixture is increased, and therefore, it is considered that the ratio in the initial mixed state cannot be maintained. Due to this, the PCR cycle number is set to desirably 28 cycles or less, more desirably 20 cycles or less. If the cycle number is about 20 cycles, bulk amplification up to about 10⁴ to 10⁵ times can be performed.

On the other hand, as shown in FIG. 2, the amount of the byproducts such as primer dimers is overwhelmingly larger than that of the target products, and also the base length of the byproducts is short, and therefore, in PCR, the efficiency of amplification of the byproducts is higher than that of the target products, and as a result, the byproducts are generated in a large excess amount. However, if the cycle number is about 20 cycles, the amount is less than several tens of ng to several μg (in the case where the amplification is started from 2 pg of mRNA, the amount of the products is only about several tens of ng or so) necessary for the preparation of a library for an analysis in the next generation sequencer described above.

Therefore, in the case where the amount of the target products contained in the reaction liquid after the nucleic acid amplification reaction is insufficient for a subsequent analysis process, by further performing a nucleic acid amplification reaction, the amount of the target products is ensured. However, in the reaction liquid, a primer dimer and the like which are byproducts are contained, and when the reaction liquid is subjected to the second nucleic acid amplification reaction as it is, the primer dimer is preferentially amplified, and the amplification of the target products is suppressed. Further, the amplification is biased, and therefore, also the ratio of the amount of the target products may be lost.

Due to this, after the reaction liquid containing the target products and the byproducts is obtained by the nucleic acid amplification reaction using nucleic acids contained in a living body-derived sample as templates, in the analysis method to which the present invention is applied, the target products and the byproducts contained in the reaction liquid are measured, and when the abundance ratio of the target products to the byproducts is lower than a prescribed value, it is determined that a process for removing the byproducts is needed, and when the abundance ratio is higher than the prescribed value, the dilution ratio of the reaction liquid after the nucleic acid amplification reaction is determined.

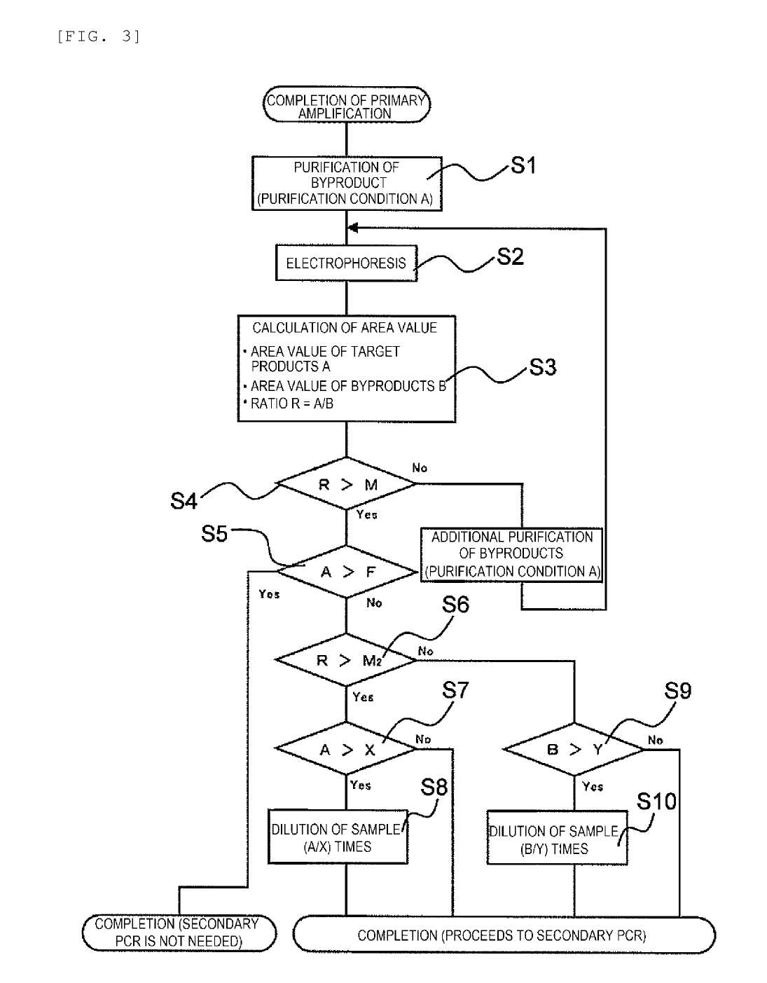

The analysis method is performed according to a flowchart shown in FIG. 3 as one example.

First, a purification process for mainly removing the byproducts from the reaction liquid after the nucleic acid amplification reaction is performed (step S1). The purification method is not particularly limited, however, a method in which a size fractionation column is used, a method in which only the target products are cut out of a gel by gel electrophoresis, and the like are exemplified. In particular, as a method with high recovery efficiency of the target products, purification using an AMPure XP solution (Beckman Coulter, Inc.) is exemplified. The method is specifically is a method in which an AMPure XP solution in an amount 0.6 times the volume of the reaction liquid is added, and only the target products are adsorbed on the bead, thereby removing the byproducts. When the amount of the AMPure XP solution to be added is increased to 0.7 times the volume of the reaction liquid, also products with a shorter base length (products with abase length of about 100 bases or more can be recovered) can be recovered, and on the other hand, when the amount of the AMPure XP solution to be added is decreased to 0.5 times the volume of the reaction liquid, also products with a longer base length (products with a base length of about 300 bases or less can be removed) can be removed. When the AMPure XP solution in an amount 0.6 times the volume of the reaction liquid is used, products with abase length of about 200 bases or more can be recovered. This is an optimal volume for separating the target products from the byproducts in the case where the base length of the cDNA which is the target product is about 250 to 8500 bases, and the base length of the byproducts is about 40 to 200 bases.

Subsequently, in order to measure the target products and the byproducts contained in the reaction liquid after the purification, electrophoresis is performed (step S2). Thereafter, from the result of the electrophoresis, the absolute amounts of the target products and the byproducts and the amount ratio thereof (for example, [target products/byproducts], however, it may be [byproducts/target products]) are measured (step S3). At this time, peak area values calculated from the electropherogram obtained as the result of the electrophoresis are determined, and from the area values, the absolute amounts of the target products and the byproducts can be determined. Specifically, a peak area (A) in a base length range of 250 to 8500 bases as the target products is calculated, and a peak area (B) in a base length range of 40 to 200 bases as the byproducts is calculated. Incidentally, it is considered that the ranges of the peaks of the byproducts and the target products vary depending on the sequence of the probe for capturing mRNA or the primer for synthesizing double-stranded cDNA, or the input amount thereof, and the ranges are not limited to the above ranges. Incidentally, it is necessary to set the boundary between the byproduct with a longest base length and the target product with a shortest base length, and it is preferred to optimize the peak areas (A) and (B) every time when the sequences of the primer and the probe are changed or when the using amounts thereof are changed.

In this analysis method, subsequently, the abundance ratio of the target products to the byproducts (for example, the value of [target products/byproducts]) and a prescribed value are compared (step S4). In the step S4, when the abundance ratio is lower than the prescribed value, it is determined that a process for removing the byproducts is needed, and when the abundance ratio is higher than the prescribed value, the process proceeds to a step for determining the dilution ratio of the reaction liquid after the nucleic acid amplification reaction. Incidentally, the phrase “determining the dilution ratio” means that the determination also includes determination that the dilution ratio is 1, that is, dilution is not needed.

That is, the case where the value of [target products/byproducts] is large means that the amount of the target products is sufficiently large with respect to the amount of the byproducts. Therefore, in the step S4, it is determined whether or not various processes can be performed using the reaction liquid containing the target products and the byproducts. Accordingly, the “prescribed value” to be used for comparison in the step S4 is a value which can be appropriately changed according to the type of an analysis process for the target products to be performed later (for example, a sequence analysis process using a next generation sequencer), a protocol thereof, a device to be used therefor.

More specifically, in the step S4, the ratio R=A/B calculated in the step S3 is compared with the prescribed value (M). In the case where (R)>(M), the ratio of the amount of the byproducts is sufficiently small with respect to the amount of the target products, and additional purification is not needed, however, in the other cases, the above-described purification is performed until the following requirement: (R)>(M) is met. The value of M is desirably 1.5 or more, more desirably 2 or more. In the step S4, in the case where it is determined that the following relationship is met: (R)>(M), by subjecting the reaction liquid containing the target products and the byproducts to a dilution process, a process for preparing a solution containing the target products and the byproducts in amounts suitable for an analysis process for the target products to be performed later is performed.

In the example shown in FIG. 3, it is premised that a sequence analysis process using a next generation sequencer is performed as the analysis process for the target products to be performed later, and for the analysis process, a given amount of the target products is needed. Therefore, it is premised that a case where a sufficient amount of the target products can be ensured and a case where a sufficient amount of the target products cannot be ensured by the above-mentioned nucleic acid amplification reaction are included. Therefore, in the example shown in FIG. 3, with respect to the reaction liquid having been determined to meet the following requirement: (R)>(M), the amount (A) of the target products and the first prescribed value (F) are compared (step S5). Then, in the step S5, the case where (A)>(F) means that a sufficient amount of the target products for the analysis process can be ensured, and it is determined that the dilution ratio is 1, that is, the reaction liquid can be used as it is. More specifically, in the step S5, the first prescribed value (F) is a value corresponding to about 100 ng or so in the case where the analysis process is a sequence determination process using a next generation sequencer. However, this value can vary depending on the flow of a subsequent step, and is not limited to the above-mentioned value.

Subsequently, in the case where it is determined that the amount (A) of the target products is insufficient in the step S5, a process for determining the dilution ratio of the reaction liquid for performing an additional nucleic acid amplification reaction (in FIG. 3, “secondary PCR”) is performed. In the example shown in FIG. 3, it is a process for ensuring a sufficient amount of the target products for the above-mentioned analysis process by the nucleic acid amplification reaction, and also for determining the dilution ratio for the purpose of reducing the amplification of byproducts as much as possible in the nucleic acid amplification reaction.

In the nucleic acid amplification reaction, in general, when a template is used in an amount not less than necessary, not only the amplification efficiency is reduced, but also a new byproduct is generated. In general, in the nucleic acid amplification reaction, it is considered that the reaction efficiency is high and also the quality of the product is high when the initial amount of the template is as small as possible. Due to this, in this example, the dilution ratio is determined so that the amount (A) of the target products becomes the upper limit value (the second prescribed value (X)) of the necessary amount for the nucleic acid amplification reaction. The value (X) corresponds to desirably 2 ng or more, more desirably 5 ng or more.

Further, also with respect to the amount (B) of the byproducts contained in the reaction liquid, the upper limit value (third prescribed value (Y)) is set. As previously described, by repeatedly performing purification using an AMPure XP solution, a molecular weight fractionation column, or the like, the byproducts can be removed to some extent, however, the target products are also lost by repetition of the purification, and therefore, the amount (B) of the byproducts sometimes cannot be sufficiently reduced when performing the additional nucleic acid amplification reaction. The value (Y) corresponds to desirably 200 pg or less, more desirably 120 pg or less.

By using the second prescribed value (X) and the third prescribed value (Y) determined as described above as indices, the dilution ratio for the reaction liquid containing the target products in an amount of (A) and the byproducts in an amount of (B) is determined (see FIG. 4). That is, for the reaction liquid for which it has been determined that the amount (A) of the target products is insufficient (as compared with (F)) in the step S5, the value of [target products/byproducts] (ratio R=A/B) calculated in the step S3 is compared with the ratio M2=[(X)/(Y)] (step S6). In the case where it is determined that the following relationship is met: (R)>(M2) in the step S6, the process proceeds to the step after the dilution ratio is determined so that the amount (A) of the target products becomes the second prescribed value (X), and in the case where it is determined that the following relationship is not met: (R)>(M2), the process proceeds to the step after the dilution ratio is determined so that the amount (B) of the byproducts becomes the third prescribed value (Y).

In the case where it is determined that the following relationship is met: (R)>(M2) in the step S6, the amount (A) of the target products is compared with the second prescribed value (X) in the step S7. Then, in the case where it is determined that the following relationship is met: (A)>(X) in the step S7, the amount of the target products in the reaction liquid is adjusted to (X) by diluting the reaction liquid to A/X times (step S8). On the other hand, in the case where it is determined that the following relationship is not met: (R)>(M2) in the step S6, the amount (B) of the byproducts is compared with the third prescribed value (Y) in the step S9. Then, in the case where it is determined that the following relationship is met: (B)>(Y) in the step S9, the amount of the byproducts in the reaction liquid is adjusted to (Y) by diluting the reaction liquid to B/Y times (step S10). Incidentally, in the case where it is determined that the following relationship is not met: (A)>(X) in the step S7, and in the case where it is determined that the following relationship is not met: (B)>(Y) in the step S9, it is determined that the dilution ratio of the reaction liquid is 1, that is, the reaction liquid is used for the additional nucleic acid amplification reaction (secondary PCR) as it is.

By performing the processes of the steps S6 to S10 as described above, the generation of the byproducts in the additional nucleic acid amplification reaction using the reaction liquid after the nucleic acid amplification reaction can be suppressed, and the target products in a desired amount necessary for a subsequent step can be produced. Incidentally, by indicating clear parameters for the processes of the steps S6 to S10, it is possible to accurately set the number of purification steps or the dilution ratio even for a sample whose initial amount is unknown.

Here, a lot of excess primers immobilized on the magnetic bead are present, which is a main cause of a byproduct, however, the present invention can also be applied under a liquid phase condition. That is, the values to be used as the indices described above can be used also for a reaction in a liquid phase state as they are as long as the sequences of the probe and the primer, and the using amounts thereof are not changed. When the sequences or the using amounts are changed, it is necessary to reset the prescribed values as previously described, however, it does not matter whether the state is a solid phase state or a liquid phase state. In addition, the target product is not limited to the cDNA library, and when a plurality of types of nucleic acids are amplified in bulk, a mixture of nucleic acids having a different base length and a different base sequence is amplified using a common primer in the same manner, and therefore, the same indices can be applied.

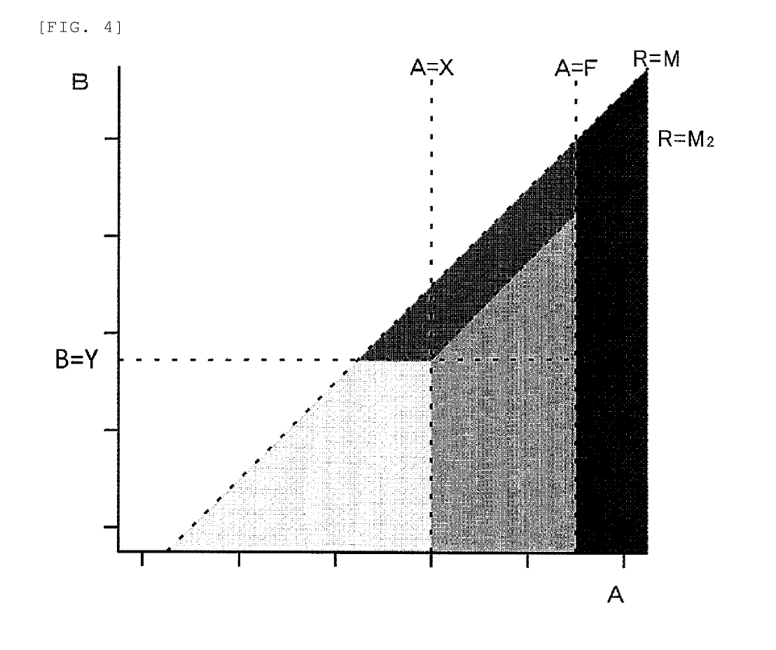

The analysis method described above can be performed using an analysis device as shown in FIG. 5 as an example. An analysis device 1 shown in FIG. 5 includes a measurement section 2 which measures the amount of the target products and the amount of the byproducts contained in the reaction liquid after performing the nucleic acid amplification reaction, and a determination section 3 which performs the process of the step S4 described above based on the value measured by the measurement section 2. According to this analysis device 1, it can be determined whether or not the reaction liquid after the nucleic acid amplification reaction can be applied to a subsequent analysis process (for example, a sequence analysis process using a next generation sequencer) for the target fragments contained in the reaction liquid, and can determine the process (for example, the steps S5 to S10) for determining the dilution ratio so that the reaction liquid can be applied to the analysis process.

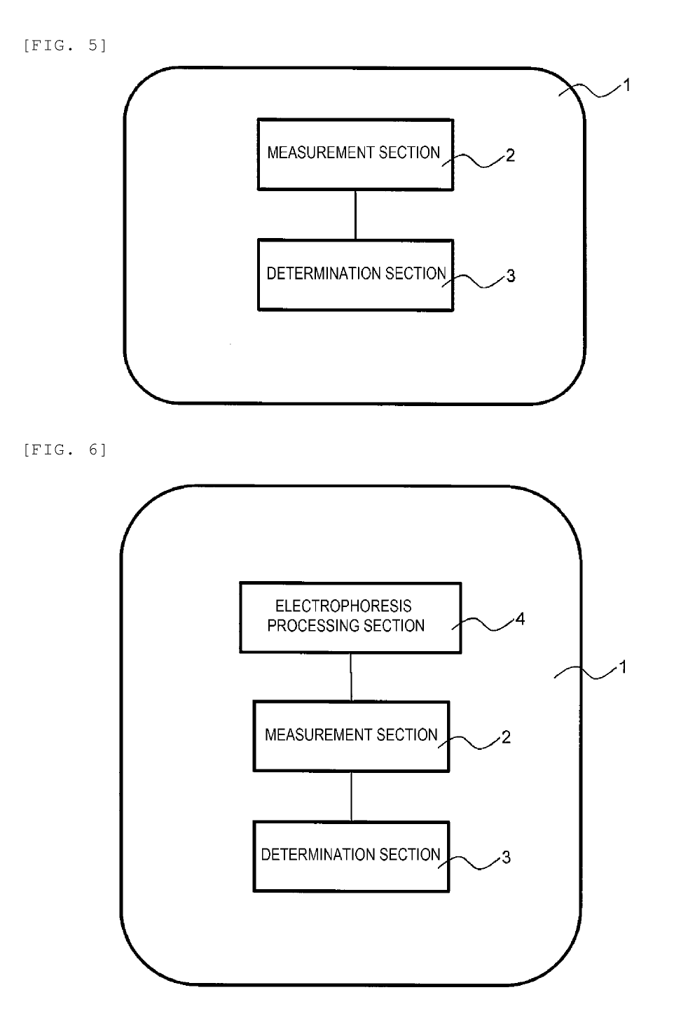

Incidentally, the analysis device 1 shown in FIG. 5 measures the amount of the target products and the amount of the byproducts in the measurement section 2 by inputting the result of electrophoresis performed for the reaction liquid after the nucleic acid amplification reaction. However, as shown in FIG. 6, this analysis device 1 may also include an electrophoresis processing section 4 which performs an electrophoresis process for the reaction liquid after performing the nucleic acid amplification reaction. According to the analysis device 1 shown in FIG. 6, the amount of the target products and the amount of the byproducts can be measured in the determination section 2 from the result of electrophoresis performed in the electrophoresis processing section 4.

Further, the determination section 3 in the analysis device 1 shown in FIG. 6 can perform a process for determining the dilution ratio based on the first prescribed value (F), the second prescribed value (X), the third prescribed value (Y), or the like, having been set according to the type of a subsequent analysis process (for example, a sequence analysis process using a next generation sequencer) for the target fragments contained in the reaction liquid, the protocol thereof, or the device to be used therefor.

The analysis device 1 configured as described above calculates the dilution ratio (also including a dilution ratio of 1) for the reaction liquid according to a subsequent analysis process (for example, a sequence analysis process using a next generation sequencer) for the target fragments contained in the reaction liquid in the determination section 3, and can output the dilution ratio. Here, the dilution ratio for the reaction liquid calculated in the determination section 3 can be output as the information which can be visually recognized by an operator who performs the dilution operation. Alternatively, the dilution ratio for the reaction liquid calculated by the determination section 3 can also be output to a dilution device capable of performing a dilution process using a given solution for the reaction liquid.

Incidentally, the analysis device 1 configured as described above can be used as a part of a reaction liquid processing device 6 which includes a dilution processing section 5 capable of performing a dilution process using a given solution for the reaction liquid based on the dilution ratio for the reaction liquid calculated by the determination section 3 as shown in FIG. 7. Although not shown in the drawing, the dilution processing section 5 includes a solution for dilution to be used for dilution, and a dispensing mechanism which dispenses the reaction liquid or the solution for dilution. Further, the dilution processing section 5 preferably includes a reagent rack for a reagent bottle filled with the reagent for dilution, a rack for a tube in which the reaction liquid has been dispensed, a tube rack to be used for dispensing the reagent, a driving device for driving the dispensing mechanism, and the like. The dilution processing section 5 of the reaction liquid processing device 6 shown in FIG. 7 appropriately selects the solution for dilution to be used for dilution according to a subsequent analysis process (for example, a sequence analysis process using a next generation sequencer) for the target products, and can dilute the reaction liquid based on the dilution ratio for the reaction liquid calculated by the determination section 3. In addition, the device can also be configured such that the dilution processing section 5 performs the process for diluting the reaction liquid by automatic control after the dilution ratio is calculated by the determination section 3 in the reaction liquid processing device 6.

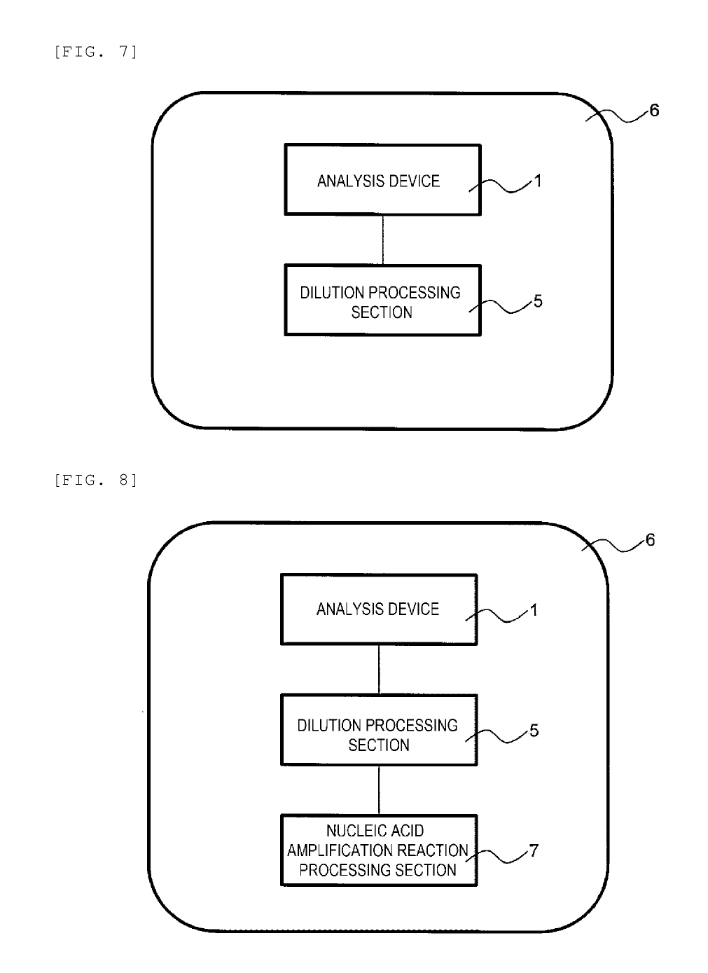

Further, the reaction liquid processing device 6 may further include a nucleic acid amplification reaction processing section 7 which performs an additional nucleic acid amplification reaction using the reaction liquid having been subjected to the dilution process in the dilution processing section 5 or the reaction liquid having not been subjected to the dilution process as shown in FIG. 8. Although not shown in the drawing, the nucleic acid amplification reaction processing section 7 includes a dispensing mechanism for adding a reagent or the like necessary for the nucleic acid amplification reaction to the reaction liquid after the dilution process, a temperature regulating device which applies a thermal cycle to the reaction liquid according to the set condition for the nucleic acid amplification reaction, and the like. In addition, the device can also be configured such that the nucleic acid amplification reaction processing section 7 performs the nucleic acid amplification reaction by automatic control after completion of the dilution operation by the dilution processing section 5.

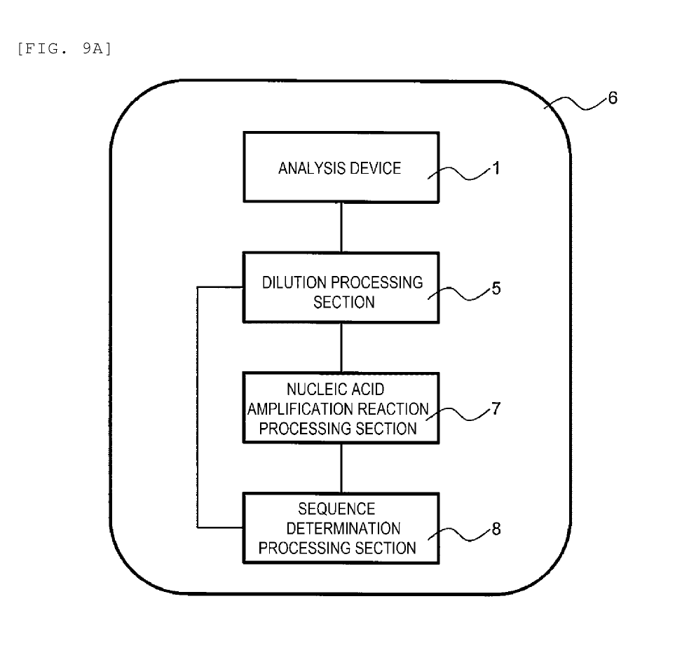

Further, the reaction liquid processing device 6 may include a processing section which performs a subsequent analysis process for the target fragments contained in the reaction liquid. As one example, in the case where a process for determining the base sequences of the target products is performed as a subsequent analysis process for the target fragments contained in the reaction liquid, as shown in FIG. 9, the reaction liquid processing device 6 can include a sequence determination processing section 8. This sequence determination processing section 8 determines the base sequences of the target amplified fragments in the reaction liquid for which it has been determined in the determination section 3 of the analysis device 1 that the dilution ratio is 1 based on the fact that the amount of the target products is sufficient for performing sequence determination or in the reaction liquid after performing the nucleic acid amplification reaction in the nucleic acid amplification reaction processing section 7. As the sequence determination processing section 8, a next generation sequencer can be used. In addition, the reaction liquid processing device 6 can also be configured such that the sequence determination processing section 8 determines the sequences of the target products by automatic control in the reaction liquid after completion of the dilution operation in the dilution processing section 5 or in the reaction liquid after completion of the nucleic acid amplification reaction in the nucleic acid amplification reaction processing section 7.

As described above, the analysis device 1 and the reaction liquid processing device 6 including the analysis device 1 are modes optimal also for an automated system. Such a system can also be configured to include, for example, a stirring device which holds a reaction plate or a tube holder and can stir a liquid in a tube by applying vibration at a given vibration level, a magnetic holder which has a plurality of magnetic pins and can insert each magnetic pin in the vicinity of each tube in the tube holder by passing the magnetic pin through the stirring device, a dispensing head which can dispense a plurality of reagents or dispense a sample or a solution for washing and also can discharge a solution in the tube, a thermostat chamber which can bring the tube holder and the stirring device therein and maintain a prescribed temperature atmosphere for a given time, a thermal cycler which can bring the tube holder therein and realize a heating and cooling cycle at temperatures under a given program, a unit which moves the tube holder between the stirring device and the thermal cycler, and an analysis section which collects a portion from a sample in the reaction tube with the dispensing head, mixes the sample with a reagent for electrophoresis, and analyzes a synthesis product of a nucleic acid in the sample by electrophoresis. By loading the above-mentioned indices to this system as parameters, when a tube containing, for example, cells is placed in the system as a start material, a mixture of nucleic acids amplified to a desired amount can be produced automatically through an optimal purification step.

### EXAMPLES

Hereinafter, the present invention will be described in more detail by way of Examples, however, the technical scope of the present invention is not limited to the following Examples.

### Example 1

A method for bulk amplification of a cDNA library prepared from a single cell for a next generation sequencer analysis will be described in detail (see FIG. 1).

Magnetic beads (diameter: 1 μm, 10⁷ beads/μL, Dynal, Inc.) coated with streptavidin were suspended to make the concentration uniform, and 120 μL of the suspension (1.2×10⁹ beads) was collected into a 2.0-mL microtube. The magnetic beads were washed three times with 120 μL of B&W buffer (1 M NaCl, 0.5 mM EDTA, 10 mM Tris (pH 8.0), 0.1% (w/v) Tween 20), and resuspended in 120 μL of the same buffer. Subsequently, 120 μL of a solution of a probe for capturing mRNA (100 pmol=6×10³ molecules) diluted with B&W buffer was prepared and added little by little to the suspension of the washed beads while mixing with a vortex.

Probe for Capturing mRNA

5′-ATATGGATCCGGCGCGCCGTCGACTTTTTTTTTTTTTTTTTTTTT
TTTVN-3′
(SEQ ID NO: 1, V = A, C, or G, N = A, C, G, or T)

The resulting mixture was reacted for 1 hour while stirring at room temperature using a thermostatic shaker (TAITEC Corporation). The supernatant was removed, and the beads were washed three times with 240 μL of B&W buffer, and then, washed three times with 240 μL of a solution containing 10 mM Tris (pH 8.0) and 0.1% (w/v) Tween 20. Finally, the beads were suspended in 120 μL of a solution containing 10 mM Tris (pH 8.0) and 0.1% (w/v) Tween 20 (1×10⁷ beads/μL). The amount of immobilized oligo per bead was 5×10⁴ molecules. In this Example, as the probe for capturing mRNA having the above sequence was used.

In this Example, 2 pg (corresponding to 1 cell) of mRNA purified from HCT116 which is a cultured human colon cancer cell line was used. A reverse transcription reaction solution 1 shown in Table 1 was prepared, and 1 μL of a solution containing 2 pg of mRNA prepared with a PBS solution was added thereto and mixed therein by pipetting.

TABLE 1
Reverse Transcription Reaction Solution 1

| Components | Volume | |
|---|---|---|
| 10x PCR buffer II (Life Technologies, Inc.) | 0.45 | μL |
| 25 mM MgCl₂ (Life Technologies, Inc.) | 0.27 | μL |
| 10% NP-40 (Pierce, Inc.) | 0.225 | μL |
| 0.1M DTT (Life Technologies, Inc.) | 0.225 | μL |
| SUPERase-In (Ambion, Inc.) | 0.045 | μL |
| RNase Inhibitor (Ambion, Inc.) | 0.045 | μL |
| UP1VN-oligo immobilized beads | 1 | μL |
| 10 mM dNTP Mix (Life Technologies, Inc.) | 0.075 | μL |
| DW | 0.665 | μL |
| Total | 3 | μL |

The resulting mixture was treated at 70° C. for 5 minutes, and then gradually cooled to 4° C. Subsequently, a reverse transcription reaction solution 2 shown in Table 2 was added thereto. After mixing by pipetting, the resulting mixture was reacted at 50° C. for 30 minutes, and then, the enzyme was inactivated at 70° C. for 5 minutes. The beads were suspended by adding 50 μL of a solution containing 10 mM Tris (pH 8.0) and 0.1% (w/v) Tween 20 thereto, and thereafter, the supernatant was removed, and the beads were washed, and then, the beads were resuspended by adding 6 μL of a solution containing 10 mM Tris (pH 8.0) and 0.1% (w/v) Tween 20 thereto.

TABLE 2
Reverse Transcription Reaction Solution 2

| Components | Volume | |
|---|---|---|
| Superscript ™ III RT (Life Technologies, Inc.) | 0.33 | μL |
| RNase Inhibitor (Ambion, Inc.) | 0.05 | μL |
| DW | 0.62 | μL |
| Total | 1 | μL |

Subsequently, a poly A tailing reaction was performed. A poly A tailing reaction solution shown in Table 3 was added to the resulting suspension and mixed therein, and thereafter, the resulting mixture was reacted at 37° C. for 15 minutes, and then, the enzyme was inactivated at 70° C. for 5 minutes. The beads were suspended by adding 50 μL of a solution containing 10 mM Tris (pH 8.0) and 0.1% (w/v) Tween 20 thereto, and thereafter, the supernatant was removed, and the beads were washed, and then, the beads were resuspended by adding 12 μL of a solution containing 10 mM Tris (pH 8.0) and 0.1% (w/v) Tween 20 thereto.

TABLE 3
Poly A Tailing Reaction Solution

| Components | Volume | |
|---|---|---|
| 10x PCR buffer II (Life Technologies, Inc.) | 0.6 | μL |
| 25 mM MgCl₂ (Life Technologies, Inc.) | 0.36 | μL |
| 100 mM dATP (Pierce, Inc.) | 0.18 | μL |
| RNaseH (Life Technologies, Inc.) | 0.3 | μL |
| Terminal deoxynucleotide transferase | 0.3 | μL |
| (Life Technologies, Inc.) | | |
| DW | 4.26 | μL |
| Total | 6 | μL |

Subsequently, a double-stranding reaction of cDNA was performed. A double-stranding reaction solution for cDNA shown in Table 4 was added to the resulting suspension and mixed therein by pipetting, and a reaction was performed using a thermal cycler under the following conditions: 95° C. for 3 min, 44° C. for 5 min, 72° C. for 6 min, and 4° C. The beads were suspended by adding 50 μL of a solution containing 10 mM Tris (pH 8.0) and 0.1% (w/v) Tween 20 thereto, and thereafter, the supernatant was removed, and the beads were washed, and then, the beads were resuspended by adding 6 μL of a solution containing 10 mM Tris (pH 8.0) and 0.1% (w/v) Tween 20 thereto.

TABLE 4
Double-Stranding Reaction Solution for cDNA

| Components | Volume |
|---|---|
| 10x EX Taq buffer (Takara Bio Inc.) | 1.9 μL |
| 2.5 mM dNTP (Takara Bio Inc.) | 1.9 μL |
| UP2VN primer* (IDT, Inc.) | 0.114 μL |
| EX Taq Hot Start Version (Takara Bio Inc.) | 0.19 μL |
| DW | 5.896 μL |
| Total | 10 μL |

*UP2VN primer:
5′-ATATCTCGAGGGCGCGCCGGATCCTTTTTTTTTTTTTTTTTTTTTTTTVN-3′
(SEQ ID NO: 2, V = A, C, or G, N = A, C, G, or T)

Subsequently, a first amplification reaction of the resulting double-stranded cDNA was performed. A first amplification reaction solution shown in Table 5 was added to the resulting suspension and mixed therein by pipetting. Thereafter, a reaction was performed using a thermal cycler under the following conditions: 95° C. for 3 min, followed by 18 cycles of 95° C. for 30 sec, 67° C. for 1 min, and 72° C. for 6 min (lengthened by 6 sec per cycle), and 72° C. for 6 min.

TABLE 5
First Amplification Reaction Solution

| Components | Volume |
|---|---|
| The above 2nd strand synthesis product | 22 μL |
| 10x EX Taq buffer (Takara Bio Inc.) | 1.9 μL |
| 2.5 mM dNTP (Takara Bio Inc.) | 1.9 μL |
| 100 μM UP1 primer* (Sigma-Genosys | 0.418 μL |
| Ltd.) | |
| EX Taq Hot Start Version (Takara Bio | 0.19 μL |
| Inc.) | |
| DW | 14.592 μL |
| Total | 41 μL |

*UP1 primer:
5′-ATATGGATCCGGCGCGCCGTCGACTTTTTTTTTTTTTTTTTTTTTTTT-3′
(SEQ ID NO: 3)

Subsequently, the first amplification product was purified. To the solution after the PCR reaction, 0.6× volume (24.6 μL) of an AMPure XP solution was added, and the resulting mixture was stirred by pipetting, and then, the beads were lightly spun down. After the mixture was left at room temperature for 5 minutes, the beads were captured with a magnet, and the supernatant was removed. To the beads, 200 μL of a 70% ethanol solution was added, and the mixture was stirred by pipetting and then left at room temperature for 30 seconds. Thereafter, the beads were captured with a magnet, and the supernatant was removed. Then, 200 μL of a 70% ethanol solution was added again to the beads, and the mixture was stirred by pipetting and then left at room temperature for 30 seconds. Thereafter, the beads were captured with a magnet, and the supernatant was removed. The beads were left at room temperature for 5 minutes, and then dried, and thereafter, 50 μL of a solution containing 10 mM Tris (pH 8.0) and 0.1% (w/v) Tween 20 was added thereto, and the beads were suspended therein by pipetting. After the resulting mixture was left at room temperature for 1 minute, the beads were captured with a magnet, and the supernatant was collected.

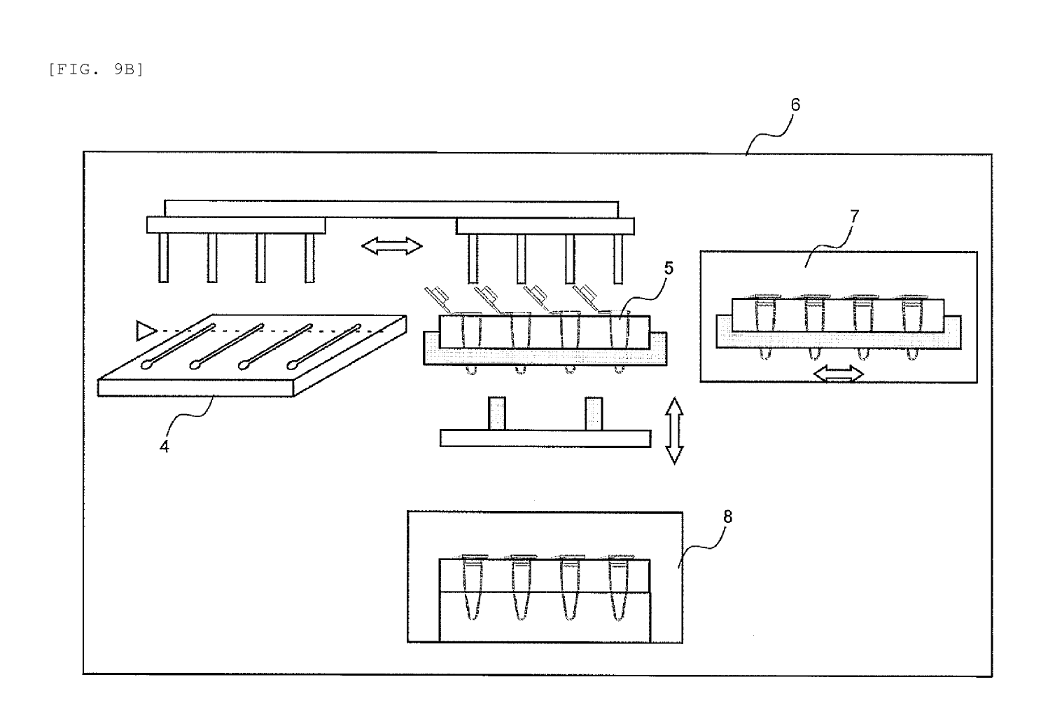

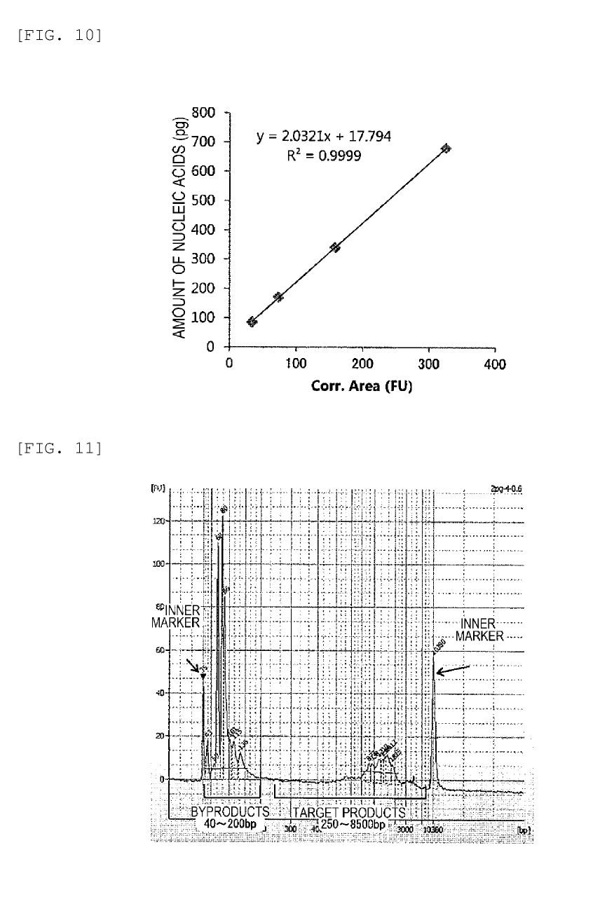

The above amplification product having been subjected to the first purification was electrophoresed by an Agilent Bioanalyzer (Agilent, Inc.). In the electrophoresis, High Sensitivity DNA Kit was used. After electrophoresis, the peak areas of the byproducts and the target products were obtained. In this Example, 2100 Expert which is an analysis software attached to the Bioanalyzer was used. In the item for Region table, the area of the byproducts was set to an area from 40 to 200 bases, and the area of the target products was set to an area from 250 to 8500 bases. The calculated values of Corr. Area were used as the peak area values, respectively. Further, in order to obtain a correlation between the peak area value and the actual molecular weight of a nucleic acid, a sample of a known concentration was electrophoresed (FIG. 10). As a result of the electrophoresis, the peak area (A) of the target products was 162.7, and the area (B) of the byproducts was 638.0 (FIG. 11). Further, R=A/B=0.26. Since the amount of the byproducts was excessively larger than that of the target products, the purification process using the 0.6× volume of the AMPure XP solution was performed again. As a result of the electrophoresis of the resulting product, the peak area (A) of the target products was 224.7, and the area (B) of the byproducts was 4.38 (FIG. 12). It was confirmed that the byproducts could be removed in a sufficient amount by purification as compared with the target products. On the other hand, the total amount of the target products was less than the target amount of 100 ng (equal to or lower than the sensitivity of a spectrophotometer, that is, 100 ng or less), and therefore, the process proceeded to a second amplification reaction.

To 1 μL of the above amplification product having been subjected to the first purification, a second amplification reaction solution shown in Table 6 was added and mixed therein by pipetting. Thereafter, a reaction was performed using a thermal cycler under the following conditions: 95° C. for 3 min, followed by 12 cycles of 95° C. for 30 sec, 67° C. for 1 min, and 72° C. for 6 min (lengthened by 6 sec per cycle), and 72° C. for 6 min.

TABLE 6
Second Amplification Reaction Solution

| Components | Volume |
|---|---|
| 10x EX Taq buffer (Takara Bio Inc.) | 5 μL |
| 2.5 mM dNTP (Takara Bio Inc.) | 5 μL |
| UP1 primer* | 0.5 μL |
| UP2 primer* (Sigma-Genosys Ltd.) | 0.5 μL |
| EX Taq Hot Start Version (Takara Bio Inc.) | 0.5 μL |
| DW | 37.5 μL |
| Total | 49 μL |

* UP1 primer:
5′-ATATGGATCCGGCGCGCCGTCGACTTTTTTTTTTTTTTTTTTTTTTTT-3′
(SEQ ID NO: 3)
* UP2 primer:
5′-ATATCTCGAGGGCGCGCCGGATCCTTTTTTTTTTTTTTTTTTTTTTTT-3′
(SEQ ID NO: 4)

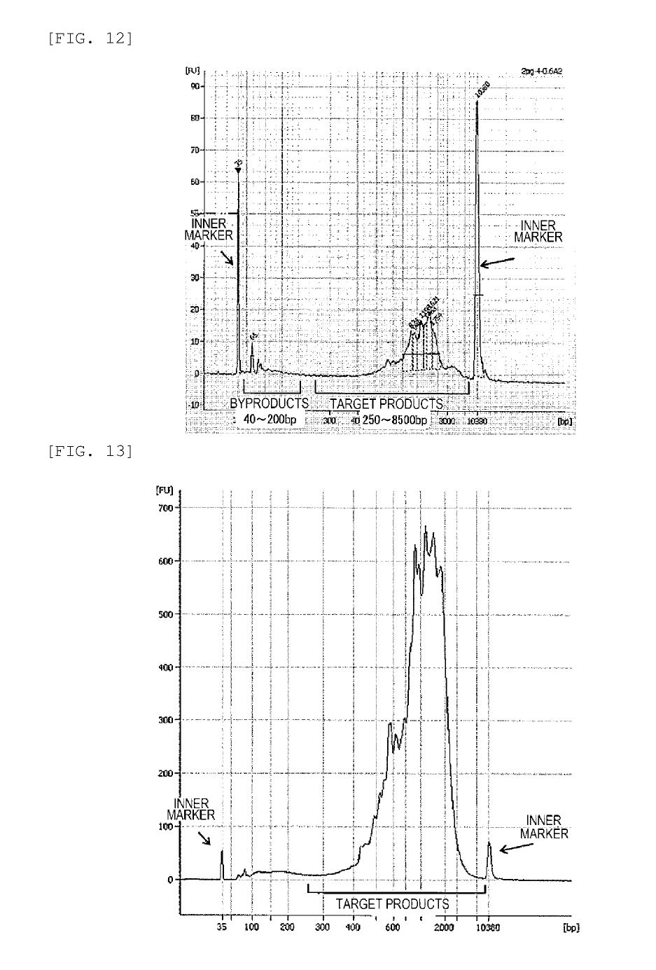

Subsequently, the second amplification product was purified. In the purification, PCR Purification Kit (JENA, Inc.) was used, and according to the protocol attached to the kit, the amplification product was eluted with 30 μL of a solution containing 10 mM Tris (pH 8.0) and 0.1% (w/v) Tween 20. The above amplification product having been subjected to the second purification was electrophoresed by an Agilent Bioanalyzer (Agilent, Inc.). In the electrophoresis, High Sensitivity DNA Kit was used. As a result, the target products could be obtained with a peak area (A) of 9526.0 which is a sufficient amount with respect to the byproducts with a peak area (B) of 302.7 (FIG. 13). The total amount of the target products was about 600 ng (in the electrophoresis, 1 μL was used), and the amplification product in an amount of 100 ng or more required for a next generation sequencer could be ensured. This case was defined as “OK”.

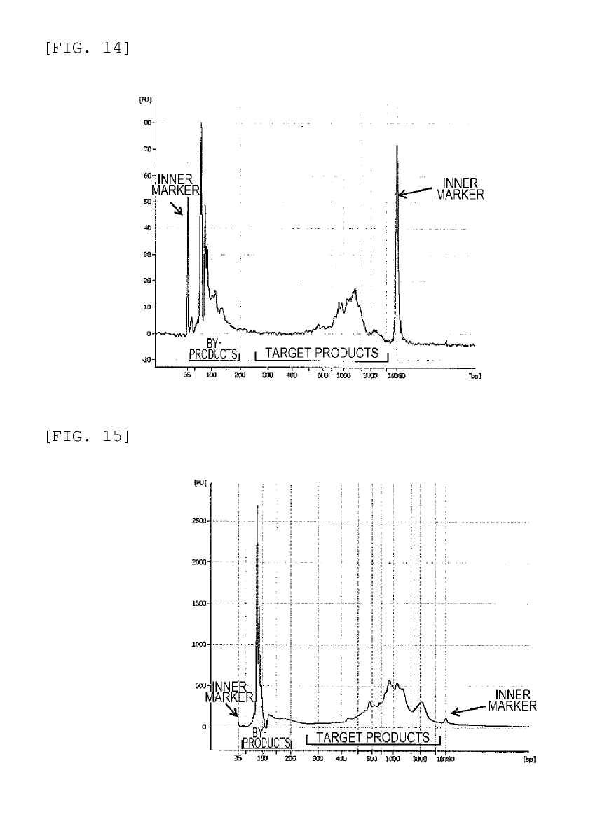

With respect to the another sample, the same experiment was performed, and the first amplification product was subjected to a purification process twice using 0.6× volume of the AMPure XP solution, and then, electrophoresis was performed in the same manner as described above. As a result, the peak area (A) of the target products was 384.5, and the area (B) of the byproducts was 230.5 (FIG. 14). The amount of the byproducts was larger as compared with the previous result, however, purification was already performed twice, and the amount of the target products was larger than the amount of the byproducts, and therefore, the process proceeded to the second amplification reaction. Also with respect to this sample, PCR Purification Kit (JENA, Inc.) was used in the same manner, and after performing purification with the kit, the resulting product was electrophoresed (FIG. 15). As a result, the byproducts were obtained with a peak area (B) of 7036.0, and the target products were obtained with a peak area (A) of 9321.9. As compared with the first case, the amount of the target products was substantially the same, however, the amount of the byproducts was very large, and although thereafter the step of removing the byproducts by purification was repeatedly performed a plurality of times, the byproducts could not be removed to the same extent as in the previous case. It is possible to further remove the byproducts by performing gel electrophoresis and cutting only the target products out of the gel, however, the possibility that the target products are lost is high, and therefore, purification by this method was not performed. In the case where the product showing such a result of the electrophoresis is analyzed using a next generation sequencer, the sequence data derived from the byproducts are mainly obtained, and the ratio the sequence data of the target products becomes very low. Due to this, this case where the sequence data derived from cDNA which is the original target cannot be obtained was defined as “NG”.

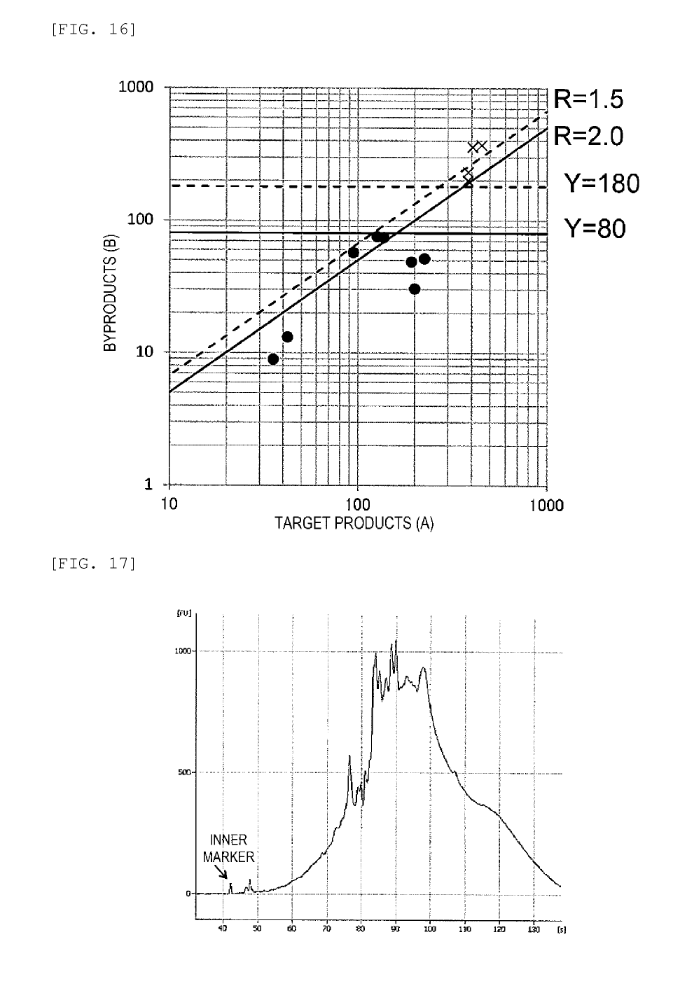

The same experiment was repeatedly performed a plurality of times, and the values (A) and (B) with respect to the cases of “OK” and “NG” were plotted (FIG. 16). As a result, in the case where the threshold R was set to 1.5 or more, the cases of “OK” and “NG” are intermingled, however, in the case where R was set as follows: R>2, the cases of “OK” could be separated from the cases of “NG”. On the other hand, as the maximum amount of the byproducts carried over to the second amplification reaction, the threshold Y was set as follows: Y=180, more desirably set as follows: Y=80.

According to this Example shown above, the R value shown in FIGS. 3 and 4 was desirably 1.5, more desirably 2, and the Y value shown in FIGS. 3 and 4 was desirably 180, more desirably 80. From these values, it became possible to clearly indicate the necessity of additional purification of the first amplification product.

### Example 2

A method for bulk amplification for a next generation sequencer analysis of a cDNA library prepared from cells, the number of which is larger than a single cell shown in Example 1, but is smaller than the commonly used size (about 10⁴ to 10⁶ cells), will be described in detail. In this Example, 200 pg (corresponding to 100 cells) of mRNA purified from HCT116 which is a cultured human colon cancer cell line was used. The procedure was allowed to proceed in the same manner as shown in Example 1 up to the first amplification.

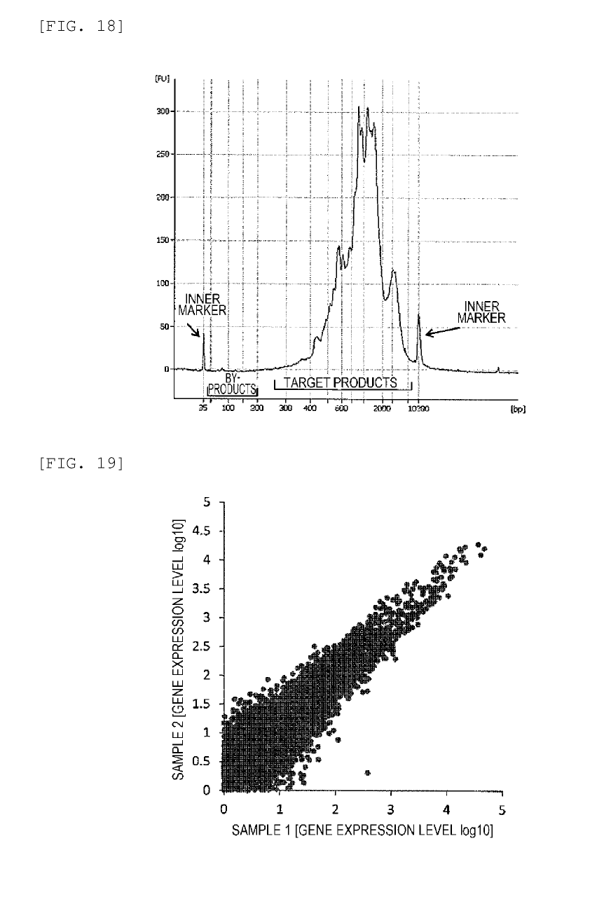

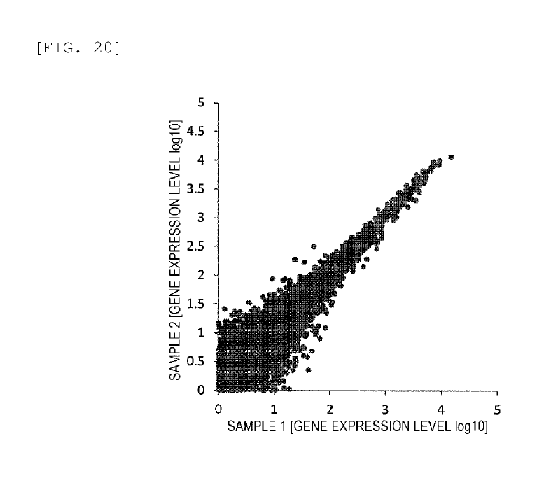

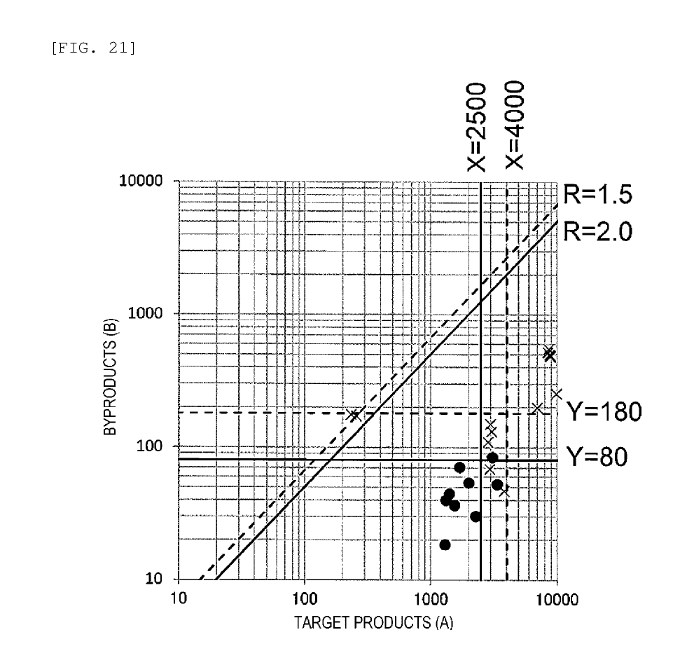

When the first amplification product was subjected to a purification process twice using 0.6× volume of the AMPure XP solution, and then, electrophoresis was performed according to the method described in Example 1, the target products could not be detected as a peak (FIG. 17). It was determined from the electrophoresis pattern that the amount of the template was too much, and therefore, the first amplification reaction was performed again using the template (that is, the double-stranded cDNA) diluted to 1/10, and the purification process using 0.6× volume of the AMPure XP solution was performed twice, and then, electrophoresis was performed. As a result, a desired pattern could be obtained (FIG. 18). Therefore, with respect to the amplification product which was not diluted (FIG. 17) and the amplification product which was diluted to 1/10 (FIG. 18), a comprehensive analysis of a mixture was performed using a next generation sequencer for two samples for each product. As a result, the reproducibility of the amplification product which was not diluted (FIG. 19) was lower than the reproducibility of the amplification product which was diluted (FIG. 20).

From this result, it was found that in the sample amplified without dilution, the abundance ratio of the mixture was lost from the result of the electrophoresis and the two analyses using the next generation sequencer. Further, the same reaction was performed also for the sample diluted to ⅕, however, the result was the same as that of the sample which was not diluted. In the same manner as described in Example 1, the sample which was not diluted and the sample which was diluted to ⅕ were determined to be “NG”, and the sample which was diluted to 1/10 was determined to be “OK”. Further, from the first electrophoresis patterns of the respective samples, (A) and (B) were obtained. The same experiment was performed a plurality of times, and the values (A) and (B) with respect to the cases of “OK” and “NG” were plotted. As a result, as the maximum amount of the target products carried over to the second amplification reaction, the threshold X was set as follows: X=4000, more desirably set as follows: X=2500.

According to this Example shown above, the X value shown in FIGS. 3 and 4 was desirably 4000, more desirably 2500. The upper limit value F of the amount (A) of the target products shown in FIG. 3 is an amount necessary for a subsequent step. In the next generation sequencer, it becomes a value corresponding to about 100 ng or so. This value is defined as about 42000 when it is converted based on FIG. 5. However, this value can vary depending on the flow of a subsequent step, and is not limited to the above-mentioned value.

As described above, according to this Example in combination with the result of Example 1, the R value, X value, Y value, and F value could be set to optimal values as shown in FIG. 21. According to this, by the flow shown in FIG. 3, it became possible to clearly indicate the necessity of additional purification of the first amplification product and the dilution ratio thereof. As a result, the generation of byproducts can be suppressed, and target products in a desired amount necessary for a subsequent step can be produced. By indicating clear parameters, it becomes possible to accurately set the number of purification steps or the dilution ratio even for a sample whose initial amount is unknown.

### REFERENCE SIGNS LIST

1: analysis device, 2: measurement section, 3: determination section, 4: electrophoresis processing section, 5: dilution processing section, 6: reaction liquid processing device, 7: nucleic acid amplification reaction processing section, 8: sequence determination processing section

1. A method for analyzing a reaction liquid after a nucleic acid amplification reaction, comprising:
measuring the amount of a target amplified fragment and the amount of amplified fragments other than the target amplified fragment after performing a nucleic acid amplification reaction using nucleic acids contained in a living body-derived sample as templates; and
determining that a process for removing the amplified fragments other than the target amplified fragment is needed when the abundance ratio of the target amplified fragment to the amplified fragments other than the target amplified fragment is lower than a prescribed value, and determining a dilution ratio of a reaction liquid after the nucleic acid amplification reaction when the abundance ratio is higher than the prescribed value.

2. The analysis method according to claim 1, wherein the nucleic acid amplification reaction is a nucleic acid amplification reaction using a carrier having an oligonucleotide composed of a poly T sequence corresponding to a poly A sequence of mRNA and a first inherent sequence immobilized thereon, the method further comprising:
capturing mRNA contained in the living body-derived sample to the carrier;
elongating a complementary strand to the mRNA from the poly T sequence;
adding a second inherent sequence to an end of the elongated strand; and
performing amplification using a first primer having a complementary sequence to the first inherent sequence and a second primer having a complementary sequence to the second inherent sequence.

3. The analysis method according to claim 1, wherein the determining further comprises determining that the dilution ratio is 1 when the amount of the target amplified fragment is higher than a first prescribed value.

4. The analysis method according to claim 1, wherein the determining further comprises:
calculating a first ratio (A/B) from a first amount (A) of the target amplified fragment and a second amount (B) of the amplified fragments other than the target amplified fragment;
calculating a second ratio (X/Y) of a second prescribed value (X) determined for the amount (A) of the target amplified fragment to a third prescribed value (Y) determined for the amount (B) of the amplified fragments other than the target amplified fragment are compared;
determining that the reaction liquid after the nucleic acid amplification reaction is diluted to (A/X) times when the first ratio (A/B) is larger than the second ratio (X/Y) and that the first amount (A) of the target amplified fragment is larger than the second prescribed value (X);
determining that the dilution ratio of the reaction liquid after the nucleic acid amplification reaction is 1 when the first ratio (A/B) is larger than the second ratio (X/Y) and that the first amount (A) of the target amplified fragment is smaller than the second prescribed value (X);
determining that the reaction liquid after the nucleic acid amplification reaction is diluted to (B/Y) times when the first ratio (A/B) is smaller than the second ratio (X/Y) and also the second amount (B) of the amplified fragments other than the target amplified fragment is larger than the third prescribed value (Y); and
determining that the dilution ratio of the reaction liquid after the nucleic acid amplification reaction is 1 when the first ratio (A/B) is smaller than the second ratio (X/Y) and also the second amount (B) of the amplified fragments other than the target amplified fragment is smaller than the third prescribed value (Y).

5. A device for analyzing a reaction liquid after a nucleic acid amplification reaction, comprising:
a measurement unit configured to measure the amount of a target amplified fragment and the amount of amplified fragments other than the target amplified fragment contained in a reaction liquid after performing a nucleic acid amplification reaction using nucleic acids contained in a living body-derived sample as templates; and
a determination unit configured to determine that a process for removing the amplified fragments other than the target amplified fragment is needed when the abundance ratio of the target amplified fragment to the amplified fragments other than the target amplified fragment is lower than a prescribed value, and determines the dilution ratio of the reaction liquid after the nucleic acid amplification reaction when the abundance ratio is higher than the prescribed value based on the value measured by the measurement unit.

6. The analysis device according to claim 5, wherein the measurement unit is configured to measure the amount of the target amplified fragment and the amount of the amplified fragments other than the target amplified fragment in the reaction liquid after the nucleic acid amplification reaction which is a nucleic acid amplification reaction using a carrier having an oligonucleotide composed of a poly T sequence corresponding to a poly A sequence of mRNA and a first inherent sequence immobilized thereon, and wherein the analysis device is further configured to:
capture mRNA contained in the living body-derived sample to the carrier;
elongate a complementary strand to the mRNA from the poly T sequence;
add a second inherent sequence to an end of the elongated strand; and
perform amplification using a first primer having a complementary sequence to the first inherent sequence and a second primer having a complementary sequence to the second inherent sequence.

7. The analysis device according to claim 5, wherein the determination unit is configured to determine that the dilution ratio is 1 when the amount of the target amplified fragment is higher than a first prescribed value.

8. The analysis device according to claim 5, wherein in the determination unit is further configured to:
calculate a first ratio (A/B) calculated from a first amount (A) of the target amplified fragment and a second amount (B) of the amplified fragments other than the target amplified fragment;
calculate a second ratio (X/Y) of a second prescribed value (X) determined for the first amount (A) of the target amplified fragment to a third prescribed value (Y) determined for the second amount (B) of the amplified fragments other than the target amplified fragment are compared;
determine that the reaction liquid after the nucleic acid amplification reaction is diluted to (A/X) times when the first ratio (A/B) is larger than the second ratio (X/Y) and that the first amount (A) of the target amplified fragment is larger than the second prescribed value (X);
determine that the dilution ratio of the reaction liquid after the nucleic acid amplification reaction is 1 when the first ratio (A/B) is larger than the second ratio (X/Y) and that the first amount (A) of the target amplified fragment is smaller than the second prescribed value (X);
determine that the reaction liquid after the nucleic acid amplification reaction is diluted to (B/Y) times when the first ratio (A/B) is smaller than the second ratio (X/Y) and that the second amount (B) of the amplified fragments other than the target amplified fragment is larger than the third prescribed value (Y); and
determine that the dilution ratio of the reaction liquid after the nucleic acid amplification reaction is 1 when the first ratio (A/B) is smaller than the second ratio (X/Y) and that the second amount (B) of the amplified fragments other than the target amplified fragment is smaller than the third prescribed value (Y).

9. A device for processing a reaction liquid after a nucleic acid amplification reaction, comprising:
the analysis device according to claim 5; and
a dilution processing unit configured to perform a dilution process for a reaction liquid after performing a nucleic acid amplification reaction according to the determination made by the determination unit in the analysis device.

10. The device for processing a reaction liquid according to claim 9, further comprising a nucleic acid amplification reaction processing unit configured to perform an additional nucleic acid amplification reaction using the reaction liquid having been subjected to the dilution process in the dilution processing unit or the reaction liquid having not been subjected to the dilution process.

11. The device for processing a reaction liquid according to claim 10, further comprising a sequence determination processing unit configured to determine the base sequence of a target amplified fragment in a reaction liquid for which it has been determined that the dilution ratio is 1 based on the determination that the amount of the target amplified fragment is higher than a first prescribed value in the determination unit in the analysis device or a reaction liquid after performing a nucleic acid amplification reaction in the nucleic acid amplification reaction processing unit.


SEQUENCE LISTING (4 sequences)

SEQ ID NO: 1 - DNA, 50 bp, Artificial
  synthetic DNA
atatggatccggcgcgccgtcgacttttttttttttttttttttttttvn

SEQ ID NO: 2 - DNA, 50 bp, Artificial
  synthetic DNA
atatctcgagggcgcgccggatccttttttttttttttttttttttttvn

SEQ ID NO: 3 - DNA, 48 bp, Artificial
  synthetic DNA
atatggatccggcgcgccgtcgactttttttttttttttttttttttt

SEQ ID NO: 4 - DNA, 48 bp, Artificial
  synthetic DNA
atatctcgagggcgcgccggatcctttttttttttttttttttttttt